(19) United States
(12) Patent Application Publication
(10) Pub. No.: US 2012/0219572 A1
(43) Pub. Date: Aug. 30, 2012

(54) Spheroidal Aggregates of Mesenchymal Stem Cells

(76) Inventors: Darwin J. Prockop, Philadelphia, PA (US); Thomas W. Bartosh, JR., Temple, TX (US); Joni Ylostalo, Belton, TX (US)

(21) Appl. No.: 13/505,379

(22) PCT Filed: Nov. 12, 2010
(86) PCT No.: PCT/US10/56502

§ 371 Date: May 1, 2012

Related U.S. Application Data
(60) Provisional application No. 61/260,559, filed on Nov. 12, 2009.

(51) Int. Cl. A61K 39/00; C12N 5/00; A61P 37/02; A61P 29/00; A61P 35/00; A61K 35/00; C12N 5/02

(52) U.S. Cl. 424/184.1; 424/93.7; 435/325; 435/383

(57) ABSTRACT

The present invention encompasses methods and compositions for reducing inflammation in a mammal. The invention includes a population of mesenchimal stromal cells that possess anti-inflammatory, anti-apoptolic, immune modulatory, and anti-tumorigenic properties.

This application claims priority based on provisional application Ser. No. 61/260,559, filed Nov. 12, 2009, the contents of which are incorporated by reference in their entirety.

### STATEMENT REGARDING FEDERALLY SPONSORED RESEARCH OR DEVELOPMENT

This invention was made, in part, using funds obtained from the U.S. Government (National Institutes of Health Grant No. P40 RR 17447), and the U.S. Government therefore has certain rights in this invention.

### BACKGROUND OF THE INVENTION

Bone marrow contains at least two types of stem cells, hematopoietic stem cells and stem cells of non-hematopoietic tissues variously referred to as mesenchymal stem cells or mesenchymal stromal cells (MSCs) or bone marrow stromal cells (BMSCs). These terms are used synonymously throughout herein. MSCs are of interest because they are easily isolated from a small aspirate of bone marrow, or other mesenchymal stem cell sources, and they readily generate single-cell derived colonies. Bone marrow cells may be obtained from iliac crest, femora, tibiae, spine, rib, knee or other mesenchymal tissues. Other sources of MSCs include embryonic yolk sac, placenta, umbilical cord, skin, fat, synovial tissue from joints, and blood. The presence of MSCs in culture colonies may be verified by specific cell surface markers which are identified with monoclonal antibodies. See U.S. Pat. Nos. 5,486,359 and 7,153,500. The single-cell derived colonies can be expanded through as many as 50 population doublings in about 10 weeks, and can differentiate into osteoblasts, adipocytes, chondrocytes (Friedenstein et al., 1970 Cell Tissue Kinet. 3:393-403; Castro-Malaspina, et al., 1980 Blood 56:289-301; Beresford et al., 1992 J. Cell Sci. 102:341-351; Prockop, 1997 Science 276:71-74), myocytes (Wakitani et al, 1995 Muscle Nerve 18:1417-1426), astrocytes, oligodendrocytes, and neurons (Azizi et al., 1998 Proc. Natl. Acad. Sci. USA 95:3908-3913); Kopen et al 1999 Proc. Natl. Acad. Sci. USA 96:10711-10716; Chopp et al., 2000 Neuroreport II 300 1-3005; Woodbury et al., 2000 Neuroscience Res. 61:364-370). In rare instances, the cells can differentiate into cells of all three germlines. Thus, MSCs serve as progenitors for multiple mesenchymal cell lineages including bone, cartilage, ligament, tendon, adipose, muscle, cardiac tissue, stroma, dermis, and other connective tissues. See U.S. Pat. Nos. 6,387,369 and 7,101,704. For these reasons, MSCs currently are being tested for their potential use in cell and gene therapy of a number of human diseases (Horwitz et al., 1999 Nat. Med. 5:309-313; Caplan, et al. 2000 Clin. Orthoped. 379:567-570).

MSCs constitute an alternative source of pluripotent stem cells. Under physiological conditions they maintain the architecture of bone marrow and regulate hematopoiesis with the help of different cell adhesion molecules and the secretion of cytokines, respectively (Clark and Keating, 1995 Ann NY Acad Sci 770:70-78). MSCs grown out of bone marrow by their selective attachment to tissue culture plastic can be efficiently expanded (Azizi et al, 1998 Proc.! Natl Acad Sci USA 95:3908-3913; Colter et al, 2000 Proc Natl Acad Sei USA 97:32 13-21 8) and genetically manipulated (Schwarz et al. 1999 Hum Gene Ther 10:2539-2549).

MSCs also are referred to as mesenchymal stem cells because they are capable of differentiating into multiple mesodermal tissues, including bone (Beresford et al., 1992 J Cell Sci 102:341-35 1), cartilage (Lermon et al., 1995 Exp Cell Res 219:211-222), fat (Beresfordet al., 1992 1 Cell Sci. 102:341-351) and muscle (Wakitani et al., 1995 Muscle Nerve 18:1417-1426). In addition, differentiation into neuron-like cells expressing neuronal markers has been reported (Woodbury et al., 2000 J Neurosci Res 61:364-370; Sanchez-Ramos et al., 2000 Exp Neurol 164:247-256; Deng et al., 2001 Biochem Biophys Res Commun 282:148-152), suggesting that MSCs may be capable of overcoming germ layer commitment.

The concept of transplantation of bone marrow has been studied by others. For example, in the Azizi, et al. reference, the investigators transplanted human bone marrow stromal cells (hBMSCs) into the brains of albino rats (Azizi, et al., 1998 Proc Natl Acad Sci USA 95:3908-3913). Their primary observations were that hBMSCs can engraft, migrate and survive in a manner similar to rat astrocytes. Further, it has been demonstrated that the bone marrow cells when implanted into the brain of adult mice can differentiate into microglia arid macroglia (Eglitis et al. Proc Natl Acad Sci USA 1997 94:4080-5).

The present invention provides the necessary data to establish that MSCs have added therapeutic benefit in treating diseases.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings certain non-limiting embodiment(s). It should be understood, however, that the invention is not limited to the precise arrangements and instrumentalities shown.

FIG. 1. is a series of images demonstrating the methodology for the generation of hMSC spheroids and the acquisition of spheroid-derived cells that maintain mesenchymal surface features; however, they can be distinguished from MSCs prepared in standard monolayer cultures by decreased expression of the surface marker PODXL and increased expression of the surface marker CD49b.

FIG. 2. is a series of images demonstrating that MSCs derived from cultured spheroids proliferate slowly but remain highly viable.

FIG. 3. is a series of images demonstrating the MSC spheroid-derived cells are significantly smaller than MSCs cultured as monolayers.

FIG. 4. is a series of images demonstrating that MSCs cultured as spheroids in hanging drops express higher levels than MSCs from monolayers of a series of therapeutic genes: TNF-α stimulated gene protein 6, or TSG-6, an anti-inflammatory protein; stanniocalcin 1, or STC-1, an anti-apoptotic protein; leukemia inhibitory factor; or LIF, a protein that regulates cell growth and development; IL-11, a protein that regulates hematopoiesis; TNF-α related apoptosis inducing ligand, or TNFSF10 (also known as TRAIL), a protein that kills some cancer cells and regulates immune response; IL-24, a protein that kills some cancer cells; CXC chemokine receptor 4, or CXCR4, a protein that regulates homing of cells; ITGA2 (also known as integrin α2), a protein involved in cell adhesion and cell signaling; and IL-8. a protein that enhances angiogenesis. The figure also illustrates that MSC spheroid-derived cells express much higher levels of the same genes that three other kinds of cells grown as spheroids, i.e. human dermal fibroblasts (hDF), a lung epithelial cancer cell line (A549), and human neural progenitor cells (hNPC).

FIG. 5. is a series of images demonstrating that production of the anti-inflammatory protein TSG-6, the anti-apoptotic protein STC-1, and the cell regulatory protein LIF is enhanced notably in MSCs derived from hanging drop cultures.

FIG. 6. is a series of images demonstrating that TNFu levels produced from LPS stimulated macrophages were decreased markedly by MSC spheroid-derived cells. The effects of the MSCs spheroid-derived cells are much greater than those of MSCs cultured as monolayers.

FIG. 7. is an image demonstrating that MSC spheroid-derived cells exhibit anti-inflammatory effects in vivo. In the experiment, peritonitis was induced in mice by injection of the irritant Zymosan, and then MSC spheroid-derived cells injected into the peritoneum. The MSC spheroid-derived cells decreases inflammation as indicated by the decrease in serum plasmin activity, a biomarker for inflammation.

FIG. 8. is a series of images demonstrating that hMSCs aggregated rapidly into three dimensional spheroids when grown in hanging drops or on a nonadherent surface.

FIG. 9. is a series of images demonstrating that hMSCs in smaller spheroids show high viability.

FIG. 10. is a series of images demonstrating that hMSC spheroids express high levels of anti-inflammatory molecule TSG-6.

FIG. 11. is an image developed from analyses by micro-arrays demonstrating that hMSC spheroids express high levels of a series of cytokines and cell adhesion molecules while the expression of cell cycle and cytoskeletal genes is down-regulated. The data demonstrated that the profile of expressed genes in hMSC spheroids is different markedly from the profile of expressed gene in hMSCs cultured as monolayers.

FIG. 12. is an image demonstrating that hMSC spheroids express high levels of molecules having therapeutic effects: TSG-6, an anti-inflammatory protein; STC1, an anti-apoptotic protein; LIF, a protein that regulates cell growth and development; IL-24, a protein that kills some cancer cells, and TRAIL, a protein that kills some cancer cells and also modulates the immune system.

FIG. 13. is a series of images demonstrating that hMSC spheroids secrete large amounts of the proteins TSG-6, STC-1, and LIF.

FIG. 14. is a series of images demonstrating that LPS stimulated macrophages secrete less TNF-α when co-cultured with hMSC spheroids. MSCs cultured as monolayers were less effective.

FIG. 15. is an image demonstrating that hMSC spheroids show the same anti-inflammatory effects as hMSC spheroid-derived cells (FIG. 6) in a mouse model of peritonitis.

FIG. 16. The expression of TSG-6 was increased as hMSCs aggregated into spheroids in hanging drops. (A) Phase contrast microscopy showing the time course of the aggregation of 25,000 hMSCs into a spheroid in a hanging drop. (Scale bar, 500 μm.) (B) H&E staining of hMSC spheroid sections from 3 day hanging drop cultures. Surface (Top), and center (Middle and Bottom) of a spheroid. (Scale bar, 50 μm.) (C) Real-time RT PCR measurements of TSG-6 expression in hMSCs shown as relative to Adh Low sample (n=3). (D) LISA measurements of TSG-6 secretion over 24 hours from hMSCs grown for 3 days at high density or as hanging drops at different cell densities (n=4). (E) Sizes of spheroids generated by hMSCs from two donors grown in hanging drops for 3 days. Sizes were measured from captured images of transferred spheroids (n=7-13). (F) Real-time RT PCR measurements of TSG-6 expression in hMSCs grown at high density or in hanging drops at 25,000 cells/drop for 1-4 days shown as relative to hMSCs grown at low density (n=3). Values are mean±SD. Abbreviations: RQ, relative quantity; Adh Low, hMSCs plated at 100 cells/cm² for 7-8 days until about 70% confluent; Adh High, hMSCs harvested from same Adh Low cultures, plated at 5,000 cells/cm² and incubated for 3 days; Sph 10k-250k, hMSCs harvested from same Adh Low cultures and incubated for 3 days in hanging drops at 10,000-250,000 cells/drop.

FIG. 17. Viability of hMSCs in spheroids. (A and B) Viability of hMSCs as determined by flow cytometry measuring PI uptake and annexin V-FITC labeling. Spheroids were dissociated with trypsin/EDTA. Representative log fluorescent dot plots and summary of the data are shown. Values are mean±SD (n=3). Abbreviations: As in FIG. 16 with 1 day to 4 day indicating days of incubation.

FIG. 18. Size analysis and i.v. infusion of spheroid hMSCs. (A) Assays of cell size by flow cytometry (n=3). hMSC sizes were estimated from forward scatter (FS) (Inset) properties of the viable population (calcein AM⁺/7AAD⁻) relative to beads with known diameters (3, 7, 15, and 25 μm). (B) Cell size assayed by microscopy. (C) Relative tissue distribution of i.v. infused hMSCs. NOD/scid mice were infused i.v. with 10⁶ monolayer or spheroid derived hMSCs. After 15 min, tissues were harvested for genomic DNA and tissue distribution of hMSCs was determined with real-time PCR for human Alu and GAPDH (n=4-5) and shown as relative to Adh High sample. *P<0.05, **P<0.01, and ***P<0.001. Values are mean±SD. Abbreviations: as in FIG. 16.

FIG. 19. Spheroid hMSCs retain the properties of hMSCs from adherent cultures. (A) Differentiation of hMSCs in osteogenic medium (Osteo Dif) and control medium (Osteo Con). Cultures were stained with Alizarin Red after 14 days. (Scale bar, 200 μm.) (B) Differentiation of hMSCs in adipogenic medium (Adipo Dif) and control medium (Adipo Con). Cultures were stained with Oil Red O after 14 days. (Scale bar, 200 μm.) (C) Growth of hMSCs (donor 2) as monolayers from high density and hanging drop cultures plated at low density (5,500 cells/plate) and passaged every 7 days (n=4). Cumulative population doublings (PDs) after each passage are shown (Inset). (D) CFU-F assays of hMSCs (donor 2) plated at 83 cells/plate and incubated for 14 days (n=4). Representative plates at passage 1 and passage 2 after transfer. Values are mean±SD. (F) Flow cytometry of surface protein expression on hMSCs. Abbreviations: as in FIG. 16 with P1 to P10 indicating passage number.

FIG. 20. Microarray assays of hMSCs from two donors. (A) Hierarchical clustering of differentially expressed genes, Genes that were either up- (236 genes) or down-regulated (230 genes) in spheroids (Sph 25k) at least twofold compared with their adherent culture counterparts (Adh Low and Adh High), were used in hierarchical clustering. The most significant Gene Ontology terms for up-regulated genes and down-regulated genes are shown next to the heat map. (B) Flow cytometry of differentially expressed surface epitopes, i.e., increased expression of CD82, a protein associated with suppression of metastases, and of CD49b, as well as downregulation of MCAM or CD146 and downregulation of ALCAM or CD166, on hMSCs. Abbreviations: as in FIG. 16.

FIG. 21. Spheroid hMSCs express high levels of anti-inflammatory and anti-tuniongenic molecules. (A) Real-time RT PCR measurements for anti-inflammatory genes (TSG-6, STC-1, and LIF), anti-tumorigenic genes (IL-24 and TRAIL), gene for an MSC horning receptor (CXCR4), and gene for the Wnt signaling inhibitor (DKK1) for two donors. Values are mean RQ±95% confidence interval from triplicate assays compared with Adh Low sample. (B) Images of high density monolayer (Adh High), spheroids (Sph 25k), and spheroid derived hMSCs (Sph 25k DC) 24 hours after transfer onto adherent (Adh) or non-adherent (Non adh) surfaces. Cultures were in six-well plates containing 1.5 ml CCM and either 200,000 hMSCs from high density cultures, eight spheroids, or 200,000 hMSCs dissociated from spheroids. After 24 hours, medium was recovered for ELISAs and cells lysed for protein assays, (Scale bar, 200 μm.) TSG-6 (C), STC-1 (D), and LIF (E) ELISAs on medium, normalized to total cellular protein. Values are mean±SD (n=3). Abbreviations: as in FIG. 16 with ND indicating not detectable and Sph 25k DC-Adh indicating hMSCs dissociated from Sph 25k and plated on cell adherent surfaces.

FIG. 22. hMSC spheroids exhibit enhanced anti-inflammatory effects in vitro and in vivo. (A) Schematic of the mouse macrophage (mMΦ) assay. mMΦs were seeded in the upper chamber of a transwell, stimulated with LPS for 90 min, the LPS was removed, and the chamber transferred to a six-well dish plated with monolayer (Adh), spheroid (Sph), or spheroid-derived hMSCs (Sph DC) at the same cell density. MΦ:hMSC (2:1). After 5 hours, medium was collected for ELISAs. (B) ELISA for mTNFα in medium from cocultures (n=3). (C-F) Anti-inflammatory activity of hMSCs in a mouse model of peritonitis. C57BL/6 mice were injected i.p. with zymosan to induce inflammation. After 15 min, the mice were injected i.p. with 1.5×10⁶ monolayer hMSCs, 60 spheroids, or 1.5×10⁶ spheroid derived cells. After 6 hours, peritoneal lavage was collected and mTNFα (C), mMPO (D), and PGE₂ (E) levels were determined with ELISAs. Total amounts of the specific molecules in the lavage are shown (n=4-8). After 24 hours, blood was collected and plasmin activity was measured from serum (n=3-6). Values are mean±SD. Not significant (NS) P≧0.05, *P<0.05, **P<0.01, and ***P<0.001. Abbreviations: as in FIGS. 16 and 21.

FIG. 23. Spheroid hMSCs maintain high viability with culture in animal product-free media and after freezing. Spheroids were cultured in the described commercially available Xeno-free medium. After 3 days, the spheroids were dissociated using trypsin/EDTA and then frozen in DMSO. The cells were thawed and the viability determined by flow cytometry measuring PI uptake and annexin V-FITC cell surface labeling. Unlabeled cells were considered viable. Abbreviations: CCM, Complete culture medium (αMEM with 17% FBS); HuSA, Human serum albumin (Baxter Healthcare); Stpro, StemPro Xeno-free medium (Gibco); Mes, Mesencult Xeno-free medium (Stem Cell Technologies).

FIG. 24. Size analysis of spheroid hMSCs cultured in animal product-free media. Flow cytometric determination of hMSC size from 3-day spheroids cultured in the described commercially available Xeno-free media. The hMSCs obtained by dissociation of the spheroids were frozen in DMSO. After a minimum of 1 week, the cells were thawed and then labeled with the viability dyes calcein AM and 7AAD to exclude dead cells from the analysis. Representative linear scatter plots of the viable population are shown. Size was quantified by comparing forward scatter (FS) properties of the cells with beads of known diameter (3, 7, 15, and 25 μm). Brackets were applied to the scatter plot at locations corresponding to the appropriate bead size (I=0, J=3, K=7, L=15, M=25 μm). Abbreviations as in FIG. 23.

FIG. 25. Analysis of therapeutic genes expressed by hMSCs cultured as spheroids in animal product-free media. Spheroids were cultured for 3 days in the defined media shown. The anti-inflammatory genes TSG-6, STC-1, and GDF-15 then were analyzed by real-time RT PCR. Values are mean RQ±SD (n=3) compared to Adh Low sample. Abbreviations as in FIG. 23.

FIG. 26. Anti-inflammatory properties of hMSCs cultured as spheroids in animal product-free media. Spheroids were cultured for 3 days in the defined media shown. The medium conditioned by the spheroids (CM) was collected, diluted 1:50, and added on mouse macrophages in presence of LPS (100 ng/ml). Macrophages cultured with (sMO) or without LPS (MO), and with non-conditioned media in presence of LPS, served as controls. After 18 hours, macrophage media were harvested and assayed for mTNFα by ELISA. Values are mean±SD (n=3). Abbreviations as in FIG. 23.

### DETAILED DESCRIPTION OF THE INVENTION

The present invention relates to the discovery that mesenchymal stromal cells (MSCs) can be manipulated in culture to possess novel therapeutic characteristics and therefore can be useful in therapy of a desired disease. Thus, in accordance with an aspect of the present invention, there are provided mesenchymal stem cells in a spheroidal aggregate or mesenchymal stem cells obtained from a spheroidal aggregate, wherein the mesenchymal stem cells express increased amounts of at least one therapeutic protein compared to mesenchymal stem cells cultured as a monolayer.

In a non-limiting embodiment, the at least one therapeutic protein is expressed by the mesenchymal stem cells in a spheroidal aggregate or by the mesenchymal stem cells obtained from a spheroidal aggregate in an amount at least 20% greater than the amount expressed by mesenchymal stem cells cultured as a monolayer. In another non-limiting embodiment, the at least one therapeutic protein is expressed by the mesenchymal stem cells in a spheroidal aggregate or by the mesenchymal stem cells obtained from a spheroidal aggregate in an amount of at least 3-fold greater than the amount expressed by mesenchymal stem cells cultured as a monolayer. In a further non-limiting embodiment, the at least one therapeutic protein is expressed by the mesenchymal stem cells in a spheroidal aggregate or the mesenchymal stem cells obtained from a spheroidal aggregate in an amount of at least 10-fold greater than the amount expressed by mesenchymal stem cells cultured as a monolayer. In yet another non-limiting embodiment, the at least one therapeutic protein is expressed by the mesenchymal stem cells in a spheroidal aggregate or by the mesenchymal stem cells obtained from a spheroidal aggregate in an amount which is at least 50-fold greater than the amount expressed by mesenchymal stem cells cultured as a monolayer. In yet another non-limiting embodiment, the at least one therapeutic protein is expressed by the mesenchymal stem cells in a spheroidal aggregate or by the mesenchymal stem cells obtained from a spheroidal aggregate in an amount which is at least 500-fold greater than the amount expressed by mesenchymal stem cells cultured as a monolayer. In yet another non-limiting embodiment, the at least one therapeutic protein is expressed by the mesenchymal stem cells in a spheroidal aggregate or by the mesenchymal stem cells obtained from a spheroidal aggregate in an amount which is at least 1,000-fold greater than the amount expressed by the mesenchymal stem cells cultured as a monolayer.

In a non-limiting embodiment, the at least one therapeutic protein is selected from the group consisting of anti-inflammatory proteins, anti-apoptotic proteins, proteins that regulate cell growth and development, proteins that regulate an immune response, proteins that regulate hematopoiesis, proteins which inhibit, prevent, or destroy the growth of tumors, proteins that regulate the homing of cells, proteins that are involved in cell adhesion and/or cell signaling, proteins that enhance angiogenesis, and combinations thereof.

For example, in non-limiting embodiments, the MSCs can be used to treat diseases associated with including but not limited to, inflammation, apoptosis of cells, immune dysregulation, including autoimmune diseases, tumors, including cancer, and the like. This is because the MSCs of the invention can be manipulated to possess therapeutic characteristics as a result of expressing increased amounts of one or more therapeutic proteins, including but not limited to an anti-inflammatory protein, an anti-apoptotic protein, a protein that regulates hematopoiesis, a protein that kills tumor cells and/or cancer cells, a protein that regulates immune response, a protein that regulates homing of cells, a protein involved in cell adhesion and/or cell signaling, a protein that enhances angiogenesis, and the like, as well as combinations thereof.

In one non-limiting embodiment, the invention provides the use of MSCs as an anti-inflammatory therapy. In another non-limiting embodiment, the invention provides the use of MSCs to decrease the programmed cell death (apoptosis) that is associated with oxygen deprivation (ischemic and hypoxia) and with multiple kinds of injury to cells and tissues. In another non-limiting embodiment, the invention provides the use of MSCs as an anti-tumor therapy, i.e., which inhibits, prevents, or destroys the growth of tumors, including cancerous tumors. In another non-limiting embodiment, the invention provides the use of MSCs for immune regulation, including, but not limited to, the treatment of autoimmune diseases.

The invention is related to the discovery that MSCs aggregate rapidly into spheroids when grown in hanging drops or on non-adherent dishes. Such MSCs are referred to herein as a “spheroidal aggregate” of MSCs or “MSC spheroids”. In a non-limiting embodiment, such MSC spheroids exhibit high viability and express high levels of one or more of a series of genes for therapeutic proteins. Examples of such proteins include, but are not limited to, TNF-α stimulated gene protein 6, or TSG-6, an anti-inflammatory protein; growth differentiation factor-15, or GDF-15, an anti-inflammatory protein; stanniocalcin-1, or STC-1, an anti-apoptotic and anti-inflammatory protein; leukemia inhibitory factor, or LIF, a protein that regulates cell growth and development; IL-11, a protein that regulates hematopoiesis; TNF-α related apoptosis inducing ligand, or TNFSF10 (also known as TRAIL), a protein that kills some cancer cells and regulates immune response; IL-24, a protein that kills some cancer cells; CD82, another protein that kills cancer cells and suppresses metastases; CXC chemokine receptor 4, or CXCR4, a protein that regulates homing of cells; ITGA2 (also known as integrin α2 or CD49b), a protein involved in cell adhesion and cell signaling; and IL-8, for a protein that enhances angiogenesis.

The present invention provides methods for pre-programming MSCs to express one or more therapeutically beneficial proteins, including but not limited to, anti-inflammatory, anti-apoptotic, immune modulatory, anti-apoptotic, and anti-tumorigenic proteins prior to the administration thereof to an animal, including human and non-human animals.

### Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent with respect to the context in which it is used.

As used herein, the term “autologous” is meant to refer to any material derived from the same individual to which it is later to be re-introduced into the individual.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can he molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures.

As used herein, the term “bone marrow stromal cells,” “stromal cells,” “mesenchymal stem cells,” “mesenchymal stromal cells” or “MSCs” arc used interchangeably and refer to a cell derived from bone marrow (reviewed in Prockop, 1997), peripheral blood (Kuznetsov et al, 2001), adipose tissue (Guilak et al., 2004), umbilical cord blood (Rosada et al., 2003), synovial membranes (De Bari et al., 2001), and periodontal ligament (Seo et al., 2005), embryonic yolk sac, placenta, umbilical cord, skin, and blood (U.S. Pat. Nos. 5,486,359 and 7,153,500), fat, and synovial fluid. MSCs are characterized by their ability to adhere to plastic tissue culture surfaces (Friedenstein et al.; reviewed in Owen & Friedenstein, 1988), and by being an effective feeder layers for hematopoietic stem cells (Eaves et al., 2001). In addition. MSCs can be differentiated both in culture and in vivo into osteoblasts and chondrocytes, into adipocytes, muscle cells (Wakitani et al., 1995) and cardiomyocytes (Fukuda and Yuasa, 2006), into neural precursors (Woodbury et al., 2000; Deng et al., 2001, Kim et al., 2006; Maresehi et al., 2006; Krampera et al., 2007), and serve as progenitors for mesenchymal cell lineages including bone, cartilage, ligament, tendon, adipose, muscle, cardiac tissue, stroma, dermis, and other connective tissues, (See U.S. Pat. Nos. 6,387,369 and 7,101,704.)

Mesenchymal stem cells (MSCs) may be purified using methods known in the art (Wakitani et al., 1995; Fukuda and Yuasa, 2006; Woodbury et al., 2000; Deng et at. 2001; Kim et at, 2006; Maresehi et al., 2006; Krampera et al., 2007).

“Graft” refers to a cell, tissue, organ or otherwise any biological compatible lattice for transplantation.

“Allogeneic” refers to a graft derived from a different animal of the same species.

“Xenogeneic” refers to a graft derived from an animal of a different species.

“Transplant” refers to a biocompatible lattice or a donor tissue, organ or cell, to be transplanted. An example of a transplant may include, but is not limited to, skin cells or tissue, hone marrow, and solid organs such as heart, pancreas, kidney, lung and liver. In one embodiment, the transplant is a human neural stem cell.

As defined herein, an “allogeneic bone marrow stromal cell (BMSC)” is obtained from a different individual of the same species as the recipient.

“Donor antigen” refers to an antigen expressed by the donor tissue to be transplanted into the recipient.

“Alloantigen” is an antigen that differs from an antigen expressed by the recipient.

As used herein, an “effector cell” refers to a cell which mediates an immune response against an antigen. In the situation where a transplant is introduced into a recipient, the effector cells can he the recipient's own cells that elicit an immune response against an antigen present in the donor transplant. In another situation, the effector cell can be part of the transplant, whereby the introduction of the transplant into a recipient results in the effector cells which are present in the transplant eliciting an immune response against the recipient of the transplant.

As used herein, a “therapeutically effective amount” is the amount of BMSCs which is sufficient to provide a beneficial effect to the subject to which the BMSCs are administered.

As used herein, “endogenous” refers to any material from or produced inside an organism, cell, or system.

“Exogenous” refers to any material introduced from or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which normally are adjacent to the fragment, e.g., the sequences adjacent to the fragment in a genome in which it naturally occurs. The term also applies to nucleic acids which have been purified substantially from other components which naturally accompany the nucleic acid, e.g., RNA or DNA or proteins, which accompany it naturally in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U’ refers to uridine.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term also should be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), and viruses that incorporate the recombinant polynucleotide.

### Description

The present invention relates to the discovery that when MSCs and in particular human MSCs (hMSCs) aggregate into spheroids, the hMSC spheroids and cells obtained from hMSC spheroids express increased levels of one or more genes encoding therapeutic proteins. Such therapeutic proteins include, but are not limited to anti-inflammatory agents, anti-apoptotic agents, proteins that regulate an immune response, proteins that regulate hematopoiesis, agents that inhibit, prevent, or destroy the growth of tumors, proteins that regulate the homing of cells, proteins that are involved in cell adhesion and cell signaling, and proteins that enhance angiogenesis, and combinations thereof. Such proteins include, but are not limited to, TSG-6, an anti-inflammatory protein; growth differentiation factor-15, or GDF-15, an anti-inflammatory protein; STC-1, an anti-apoptotic and anti-inflammatory protein; LIF, a protein that regulates cell growth and development; IL-11, a protein that regulates hematopoiesis: TNF-α a related apoptosis inducing ligand, or TNFSF 10 (also known as TRAIL), a protein that kills some cancer cells and regulates immune response; IL-24, a protein that kills some cancer cells; CD82, another protein that kills cancer cells and suppresses metastases; CXC chemokine receptor 4, or CXCR4, a protein that regulates homing of cells; ITGA2 (also known as integrin α2 or CD49b), a protein involved in cell adhesion and cell signaling; and IL-8, a protein that enhances angiogenesis. The disclosure presented herein demonstrates that hMSC spheroids and hMSCs obtained from hMSC spheroids exhibit anti-inflammatory effects in an in vitro inflammation assay and are anti-inflammatory in an in vivo mouse model of peritonitis. Accordingly, hMSC spheroids and hMSCs obtained from hMSC spheroids are useful as anti-inflammatory therapy in many diseases. In some instances, hMSC spheroids and hMSCs obtained from hMSC spheroids are useful as anti-tumor therapy in many cancers.

The present invention encompasses methods and compositions for reducing and/or eliminating an inflammatory response in a mammal by treating the mammal with an amount of MSC spheroids, or cells obtained from hMSC spheroids, effective to reduce or inhibit inflammation in the mammal.

The present invention provides methods of pre-programming MSCs to express increased amounts of therapeutic proteins, including but not limited to, therapeutically beneficial anti-inflammatory, anti-apoptotic, immune regulatory, and anti-tumorigenic proteins prior to the administration or transplantation thereof. In some instances, the method includes activating MSCs and administer MSCs so that anti-inflammatory, anti-apoptotic, immune modulatory, and/or anti-tumorigenic proteins are at maximal expression when administered to a patient.

### Therapy to Inhibit Inflammation and/or Anti-Tumor Therapy

The present invention includes a method of using MSC spheroids or cells obtained from hMSC spheroids as a therapy to inhibit or modulate inflammation. The invention is based on the discovery that MSCs when aggregated rapidly form spheroids. The hMSC spheroids exhibit high viability and express high levels of anti-inflammatory, anti-apoptotic, immune modulatory, and/or anti-tumorigenic molecules. In some instances, the hMSC spheroids or cells obtained from hMSC spheroids secrete high levels of the anti-inflammatory proteins. The hMSC spheroids or cells obtained from hMSC spheroids also exhibited anti-inflammatory effects in an in vitro inflammation assay and in a mouse model of peritonitis.

One skilled in the art would appreciate, based upon the disclosure provided herein, that the ability of hMSC spheroids or cells obtained from hMSC spheroids to suppress inflammation provides a means for an anti-inflammatory therapy.

Based upon the disclosure provided herein, MSCs can be obtained from any source. The MSCs may be autologous with respect to the recipient (obtained from the same host) or allogeneic with respect to the recipient. In addition, the MSCs may be xenogeneic to the recipient (obtained from an animal of a different species); for example, rat MSCs may be used to suppress inflammation in a human.

In a further embodiment, MSCs used in the present invention can be isolated, from the bone marrow of any species of mammal, including but not limited to, human, mouse, rat, ape, gibbon, bovine. In a non-limiting embodiment, the MSCs are isolated from a human, a mouse, or a rat. In another non-limiting embodiment, the MSCs are isolated from a human.

Based upon the present disclosure, MSCs can be isolated and expanded in culture in vitro to obtain sufficient numbers of cells for use in the methods described herein provided that the MSCs are cultured in a manner that promotes aggregation and formation of spheroids. For example, MSCs can be isolated from human bone marrow and cultured in complete medium (DMEM low glucose containing 4 mM L-glutamine, 10% PBS, and 1% penicillin/streptomycin) in hanging drops or on non-adherent dishes: however, the invention should in no way he construed to be limited to any one method of isolating or to any culturing medium. Rather, any method of isolating and any culturing medium should be construed to be included in the present invention provided that the MSCs are cultured in a manner that promotes aggregation and formation of spheroids.

Any medium capable of supporting MSCs in vitro may be used to culture the MSCs. Media formulations that can support the growth of MSCs include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (PBS) or 1-20% horse serum is added to the above medium in order to support the growth of MSCs. A defined medium, however, can also be used if the growth factors, cytokines, and hormones necessary for culturing MSCs are provided at appropriate concentrations in the medium. Media useful in the methods of the invention may contain one or more compounds of interest, including but not limited to antibiotics, mitogenic or differentiation compounds useful for the culturing of MSCs. The cells may be grown, in one non-limiting embodiment, at temperatures between 27° C. to 40° C., in another non-limiting embodiment, at 31° C. to 37° C., and in another non-limiting embodiment, in a humidified incubator. The carbon dioxide content may be maintained between 2% to 10% and the oxygen content may be maintained between 1% and 22%. However, the invention should in no way be construed to be limited to any one method of isolating and culturing MSCs. Rather, any method of isolating and culturing MSCs should be construed to be included in the present invention.

Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml.

In general, the mesenchymal stem cells are cultured under conditions which, as noted hereinabove, provide for the aggregation of the mesenchymal stem cells into a spheroidal aggregate, and provide for optimal expression of the therapeutic protein(s).

In one non-limiting embodiment, the mesenchymal stem cells are cultured in a medium, such as complete culture medium (CCM), for example, which includes serum in an amount effective to upregulate one or more of the hereinabove noted therapeutic proteins. For example, the medium may include fetal bovine serum in an amount of up to 20%. In a non-limiting embodiment, the fetal bovine serum is present in an amount of about 17%. The mesenchymal stem cells are cultured under conditions and for a period of time (for example, 7 or 8 days) sufficient to provide a sufficient number of cells for further culturing. The culture medium may include growth factors other than or in addition to serum to upregulate one or more of the hereinabove noted therapeutic proteins.

The cells then are cultured under conditions which promote the formation of spheroidal aggregates of the cells. In one non-limiting embodiment, the cells are cultured as hanging drops. Each drop of cells contains mesenchymal stem cells in an amount which provides for optimal expression of the at least one therapeutic protein. In a non-limiting embodiment, the hanging drops of the cells are cultured in a medium, such as complete culture medium, containing fetal bovine serum in an amount of up to 20%. In a non-limiting embodiment, the fetal bovine serum is present in an amount of about 17%.

In another non-limiting embodiment, each hanging drop of mesenchymal stem cells that is cultured contains from about 10,000 to about 500,000 cells/drop. In another non-limiting embodiment, each hanging drop of mesenchymal stem cells that is cultured contains from about 10,000 to about 250,000 cells/drop. In a further non-limiting embodiment, each hanging drop of cells contains from about 10,000 to about 25,000 cells/drop. In yet another non-limiting embodiment, each hanging drop of cells contains about 25,000/drop.

The hanging drops of mesenchymal stem cells are cultured for a period of time sufficient for forming spheroidal aggregates of the mesenchymal stem cells. In general, the drops of cells are cultured for a period of time of up to 4 days.

Once the spheroidal aggregates of the mesenchymal stem cells are formed, the mesenchymal stem cells may, if desired, be dissociated from the spheroids by incubating the spheroids in the presence of a dissociation agent, such as trypsin and/or EDTA, for example.

The spheroidal aggregates of mesenchymal stem cells, or mesenchymal stem cells derived from the spheroidal aggregates may be administered to an animal to provide a desired therapeutic effect. The animal may be a mammal, including but not limited to, human and non-human primates.

Thus, another embodiment of the present invention encompasses administering MSCs to the recipient of a transplant. MSCs can be administered by a route which is suitable for the placement of the transplant, i.e. a biocompatible lattice or a donor tissue, organ or cell, to be transplanted. MSCs can be administered systemically, i.e., parenterally, by intravenous injection or can be targeted to a particular tissue or organ, such as bone marrow. MSCs can be administered via a subcutaneous implantation of cells or by injection of the cells into connective tissue, for example, muscle.

MSCs can be suspended in an appropriate pharmaceutical carrier or diluent. Suitable excipients for injection solutions are those that are biologically and physiologically compatible with the MSCs and with the recipient, such as buffered saline solution or other suitable excipients. The composition for administration can be formulated, produced and stored according to standard methods complying with proper sterility and stability.

The dosage of the MSCs varies within wide limits and may be adjusted to the individual requirements in each particular case. The number of cells used depends on the age, weight, sex, and condition of the recipient, the number and/or frequency of administrations, the disease or disorder being treated, and the extent or severity thereof, and other variables known to those of skill in the art.

### Advantages of Using MSCs

Based upon the disclosure herein, it is envisioned that the MSCs of the present invention can be used in conjunction with current modes, for example the use of anti-inflammatory therapy, for the treatment diseases, disorders, or conditions associated with inflammation. An advantage of using MSCs in place of or in conjunction with anti-inflammatory agents is that by using the methods of the present invention to meliorate the severity of inflammation in the recipient, the amount of anti-inflammatory agents used and/or the frequency of administration of anti-inflammatory agents can be reduced. A benefit of reducing the use of anti-inflammatory agents is the alleviation of unwanted side effects associated with anti-inflammatory agents. It is also contemplated that the cells of the present invention may be administered into a recipient as a “one-time” therapy for the treatment of inflammation. A one-time administration of MSCs into the recipient eliminates the need for chronic anti-inflammatory therapy. If desired, however, multiple administrations of MSCs may also be employed.

The invention described herein also encompasses a method of preventing or treating inflammation by administering MSCs in a prophylactic or therapeutically effective amount for the prevention, treatment or amelioration of inflammation. An effective amount of MSCs can be determined by comparing the level of inflammation in a recipient prior to the administration of MSCs thereto, with the level of inflammation present in the recipient following the administration of MSCs thereto. A decrease, or the absence of an increase, in the level of inflammation in the recipient with the administration of MSCs thereto, indicates that the number of MSCs administered is a therapeutic effective amount of MSCs.

Based upon the disclosure herein, it is envisioned that the MSCs of the present invention can he used in conjunction with current modes, for example the use of anti-tumor therapy, for the treatment of cancer. Cancers which may be treated by the mesenchymal stem cells of the present invention include, but are not limited to, lung cancer, Kaposi's sarcoma, colorectal cancer, glioma, breast cancer, including breast metastases, melanoma, including melanoma metastases, hepatomas, pancreatic cancer, and osteosarcomas. An advantage of using MSCs in place of or in conjunction with anti-tumor agents is that by using the methods of the present invention to ameliorate the severity of cancer in the recipient, the amount of anti-tumor agents used and/or the frequency of administration of anti-tumor agents can be reduced. A benefit of reducing the use of anti-tumor agents is the alleviation of unwanted side effects associated with anti-tumor agents.

It also is contemplated that the cells of the present invention may be administered into a recipient as a “one-time” therapy for the treatment of cancer. A one-time administration of MSCs into the recipient eliminates the need for chronic anti-tumor therapy. If desired, however, multiple administrations of MSCs may also be employed.

The invention described herein also encompasses a method of preventing or treating cancer by administering MSCs in a prophylactic or therapeutically effective amount for the prevention, treatment or amelioration of cancer. An effective amount of MSCs can be determined by comparing the level of cancer in a recipient prior to the administration of MSCs thereto, with the level of cancer present in the recipient following the administration of MSCs thereto. A decrease, or the absence of an increase, in the level of cancer in the recipient with the administration of MSCs thereto, indicates that the number of MSCs administered is a therapeutic effective amount of MSCs.

When used in transplantation, mesenchymal stem cells are capable of systemic migration, are not prone to tumor formulation, and appear to tolerize the immune response across donor mismatch. Thus, based upon the disclosure herein, it is envisioned that the MSCs of the present invention can be used in conjunction with current modes, for example the use of immune modulation therapy for graft versus host disease following transplants of bone marrow or organs or to treat or prevent transplant rejection, or for autoimmune diseases such as lupus and autoimmune related diseases such as Type I diabetes, rheumatoid arthritis, thyroiditis, and psoriasis, and autoproliferative diseases.

Based upon the disclosure herein, it is envisioned that the MSCs of the present invention can be used in conjunction with current modes, for example the use of therapies to limit programmed cell death as occurs following injury to tissues from lack of oxygen (ischemia or hypoxia) or in diseases such as Alzheimer's disease, parkinsonism, and other neurodegenerative diseases, as well as stroke, brain trauma, or concussion.

It also is contemplated that within the scope of the present invention that the mesenchymal stem cells in a spheroidal aggregate or the mesenchymal stem cells obtained from a spheroidal aggregate, may, in addition to the treatments described hereinabove, be used in other therapies employing mesenchymal stem cells, with the added advantage that the mesenchymal stem cells of the present invention express increased amounts of one or more of the therapeutic proteins hereinabove described. For example, the mesenchymal stem cells of the present invention may be administered to an animal, whereby such mesenchymal stem cells differentiate into a desired cell type and/or generate or regenerate a desired tissue in an animal. For example, the mesenchymal stem cells of the present invention may be administered to an animal, whereby such mesenchymal stem cells may differentiate into cells such as osteocytes, adipocytes, chondrocytes, myocytes, astrocytes, oligodendrocytes, neurons, and/or may generate bone, cartilate, ligaments, tendons, adipose tissue, muscle, cardiac tissue, stroma, dermal tissue, and/or other connective tissues in the animal. Thus the mesenchymal stem cells of the present invention may be used to provide an animal with any of a plurality of desired cell types, and/or generate or regenerate any of a plurality of desired tissues in an animal, while providing the animal with the therapeutic proteins hereinabove described.

For example, the mesenchymal stem cells in a spheroidal aggregate, or the mesenchymal stem cells obtained from a spheroidal aggregate, may be administered to an animal to repair or regenerate bone, tendons, and/or cartilage, including chondrogenesis/knee and joint repair, or may be used as an adjunct therapy through protein production and immune mediation. The mesenchymal stem cells in a spheroidal aggregate, or the mesenchymal stem cells obtained from a spheroidal aggregate, may be used for the in vivo production of cytokines for the support of cotransplanted hematopoietic stem cells, and for producing enzymes that are deficient in animal models of lysosomal storage disorders.

In addition, the mesenchymal stem cells in a spheroidal aggregate, or mesenchymal stem cells obtained from a spheroidal aggregate, may be used to regenerate cardiac tissue and/or effect revascularization of cardiac tissue following myocardial infarction, as well as for repair of intervertebral disc defects and spine therapy, repair of tissue or blood vessel damage caused by stroke, therapy for epilepsy, and skeletal tissue repair.

The mesenchymal stem cells in a spheroidal aggregate, or mesenchymal stem cells obtained from a spheroidal aggregate, also may be employed in treating wounds. The mesenchymal stem cells in a spheroidal aggregate, or mesenchymal stem cells obtained from a spheroidal aggregate, may be administered systemically, or may be applied to the wound topically. Upon entering the wound, the mesenchymal stem cells in a spheroidal aggregate, or mesenchymal stem cells obtained from a spheroidal aggregate, interact with other wound cells through paracrine mechanisms, and interaction with vascular endothelial cells and immuno-modulation accelerate wound healing and reduce scar formation upon completion of the healing process.

In another non-limiting embodiment, the mesenchymal stem cells in spheroidal aggregates, or mesenchymal stem cells obtained from spheroidal aggregates, may be employed in treating diseases or disorders of the lung. Although the scope of this embodiment is not to be limited to any theoretical reasoning, it is believed that mesenchymal stem cells “home” to areas of diseased or damaged lung tissue, whereby the mesenchymal stem cells may replace or repair the damaged or diseased lung tissue. Diseases or disorders of the lung which may be treated with the mesenchymal stem cells in spheroidal aggregates, or mesenchymal stem cells obtained from spheroidal aggregates include, but are not limited to, lung cancer, cystic fibrosis, α1-anti-trypsin deficiency, and idiopathic pulmonary fibrosis, or IPF.

In another non-limiting embodiment, the mesenchymal stem cells in a spheroidal aggregate, or mesenchymal stem cells obtained from a spheroidal aggregate, may be employed in treating brain injuries or disorders, and in repairing and/or regenerating brain tissue, as well as repairing and/or regenerating blood vessels in the brain, or promoting angiogenesis in the brain. For example, the mesenchymal stem cells in a spheroidal aggregate, or mesenchymal stem cells obtained from a spheroidal aggregate, may be used in repairing and/or regenerating brain tissues, and/or repairing and/or regenerating blood vessels in the brain which have been damaged as a result of a stroke or a concussion.

In addition, the mesenchymal stem cells in a spheroidal aggregate, or mesenchymal stem cells obtained from a spheroidal aggregate, when administered, escape trapping in the lung, thereby providing an advantage with respect to treating diseases in distal organs, including cancer and other diseases or disorders hereinabove described.

Furthermore, by culturing the mesenchymal stem cells as spheroidal aggregates, the mesenchymal stem cells become “pre-activated,” i.e., the mesenchymal stem cells in spheroidal aggregates, or mesenchymal stem cells obtained from spheroidal aggregates, express in vivo the increased amounts of the one or more therapeutic proteins described herein immediately upon administration to an animal, as opposed to delayed expression of the therapeutic protein(s) in the animal, e.g., at about 10 to 24 hours after administration of mesenchymal stem cells which are not in the form of spheroidal aggregates or not obtained from spheroidal aggregates.

The mesenchymal stem cells in spheroidal aggregates, or mesenchymal stem cells obtained from spheroidal aggregates, in a non-limiting embodiment may be administered alone, or in combination with drugs or other pharmaceutical agents known to be employed in the treatments hereinabove described.

In another non-limiting embodiment, the mesenchymal stem cells in spheroidal aggregates, or mesenchymal stem cells obtained from a spheroidal aggregate, may be genetically engineered with an exogenous polynucleotide encoding a therapeutic agent. Such polynucleotide may be contained in an appropriate expression vector such as those hereinabove described, and such vector may be introduced into the mesenchymal stem cells of the present invention by means known to those skilled in the art. Thus, such mesenchymal stem cells may be employed in gene therapy treatments, with the advantage that such genetically engineered mesenchymal stem cells express increased amounts of one or more therapeutic agents.

Applicants also have discovered that media in which spheroidal aggregates have been cultured may provide a therapeutic effect as hereinabove described. Thus, in another aspect of the present invention, there is provided a method of providing a therapeutic effect in an animal, comprising administering to the animal a composition comprising a medium in which there have been cultured previously spheroidal aggregates of mesenchymal stem cells. The composition includes the medium in an amount effective to provide a therapeutic effect in said animal. The therapeutic effects may be those hereinabove described, including, but not limited to, an anti-inflammatory effect, an anti-tumor effect, or the regulation of an immune response.

Such medium, in a non-limiting embodiment, may be prepared by culturing mesenchymal stem cells in a medium, such as those hereinabove described, which promotes the aggregation and formation of spheroids of mesenchymal stem cells, and under conditions and for a period of time, such as hereinabove described, which also promote the formation of spheroidal aggregates of mesenchymal stem cells. Once the spheroidal aggregates of mesenchymal stem cells are formed, the mesenchymal stem cells are dissociated from the spheroids as hereinabove described, and are separated from the medium.

The medium, also referred to as a conditioned medium, then may be administered to an animal in an amount effective to provide a desired therapeutic effect in the animal. In a non-limiting embodiment, the conditioned medium is administered in conjunction with an appropriate pharmaceutical carrier or diluent, such as, for example, buffered saline solution or other excipients as hereinabove described. The composition can be formulated, produced, and stored according to standard methods complying with proper sterility and stability.

The dosage of the conditioned medium varies within wide limits and may be adjusted to the individual requirements in each particular case. The amount of media to be administered depends on the age, weight, sex, and condition of the recipient, the number and/or frequency of administrations, the disease or disorder being treated, and the extent or severity thereof, and other variables known to those skilled in the art.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### Unique Characteristics of Human Mesenchymal Stromal Cells Derived from Multicellular Spheroid Cultures

Human mesenchymal stromal cells (hMSCs) show great promise for the repair of damaged and dysfunctional tissues. Our preliminary studies showed that hMSCs cultured as hanging drops aggregated into spheroids with anti-inflammatory propensities. In the current work we sought to define the unique characteristics of the spheroid derived cells (SDCs) in comparison to hMSC monolayer cultures and spheroids derived from other cell types. To obtain SDCs for further studies, spheroids were dissociated with trypsin/EDTA and the cells were harvested by centrifugation. By microscopy the measured diameter of SDCs was ½ and the volume approximately ¼ of the monolayer hMSCs. Interestingly, the SDC size was independent of the initial cell number within the hanging drop, and therefore, spheroid size. High SDC viability also was confirmed by microscopy as well as flow cytometry trypan blue and Annexin/PI. Moreover, SDCs maintained their mesenchymal characteristics as determined by flow cytometry (CD73+, CD90+, CD105+) and CFU-F assays; however, SDC expression of CD49b, was increased, while PODXL, CD49d, and CD49f were decreased. Surprisingly, hMSC spheroids did not grow, nor did the cells divide significantly (less than 3% in S-phase) within these aggregates similar to fibroblasts, but in contrast to neurospheres and cancer spheres. Also in contrast to other sphere types, anti-tumorigenic (TRAIL and IL-24) and anti-inflammatory (TSG-6, STC-1, and LIF) genes were up-regulated in hMSC spheroids. The elevated secretion of these anti-inflammatory factors in hMSC SDCs was measured with ELISAs and the anti-inflammatory properties were demonstrated using a co-culture system with LPS activated macrophages and in a mouse model of peritonitis. In conclusion, we determined distinct features of hMSC SDCs in terms of morphology, phenotype, growth, viability, and anti-inflammatory characteristics. These results suggest that SDCs are unique pre-activated small cells that could show beneficial effect in regenerative therapies.

### Methodology for the Generation of hMSC Spheroids and the Acquisition on of Spheroid-Derived Cells That Maintain Mesenchymal Surface Features

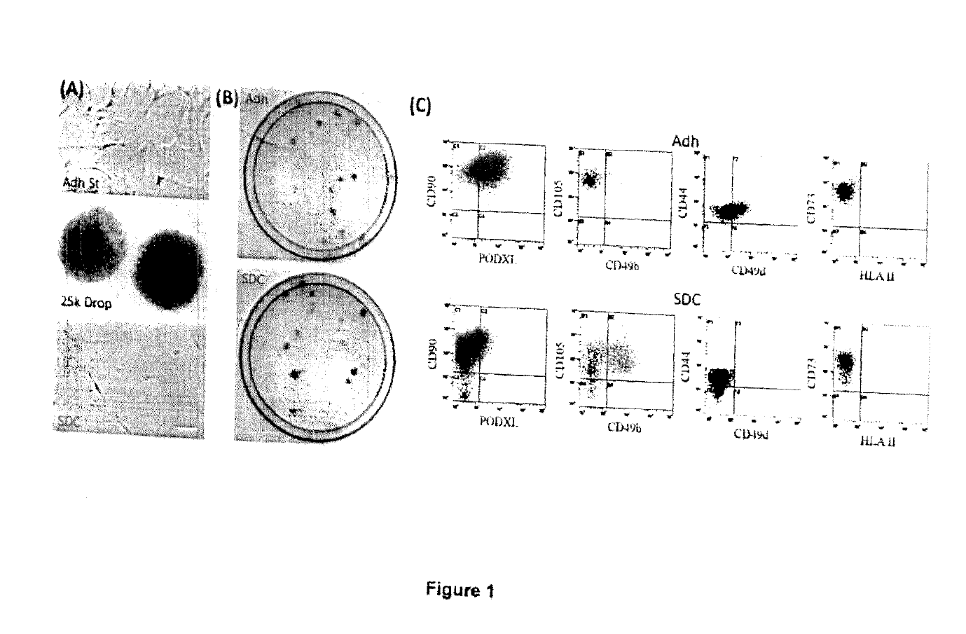

To obtain hMSCs, nucleated cells were isolated from bone marrow aspirates by density gradient centrifugation and resuspended in complete culture medium (CCM): α-MEM, 17% FBS, penicillin/streptomycin, L-glutamine. Nucleated cells were plated and after 24 hours non-adherent cells were discarded. Adherent cells were expanded until approximately 70% confluent, harvested with trypsin/EDTA, and replated at 50 cells/cm³ in cell factories. The cells were expanded until 70% confluent then frozen as passage 1 cells. Frozen vials of passage 1 MSCs were thawed, plated at 100 cells/cm², and further expanded for 7 days prior to freezing. In this study, passage 1 or 2 frozen MSCs were harvested and grown at 100 cells/cm² for or 7-8 days (Adh St) before harvesting for various assays. (FIG. 1A) To generate multi-cellular spheroids, MSCs were plated as hanging drops on the lid of a culture dish at 25,000 cells/drop (25k Drop) in 35 μl of CCM for 3 days. To acquire spheroid-derived cells (SDCs), MSC spheroids were harvested by scraping and dissociated with trypsin/EDTA for 5-10 min (FIG. 1B) CFU assays were performed on SDCs, and MSCs derived from adherent monolayers (Adh), plating the cells at 1 cell/cm² and culturing in CCM for 14 days. The cells were fixed in methanol and labeled 5 min with crystal violet. Images were captured on the Bio-Rad VersaDoc imaging system. (FIG. 1C) For FCM analysis, SDCs were labeled with the antibodies shown for 20-30 min at RT. Cells were then washed in PBS and surface labeling measured on a Beckman Coulter FC500 benehtop analyzer. Scale bar=50 μm.

### MSCs Derived from Cultured Spheroids Proliferate Slowly but Remain Highly Viable

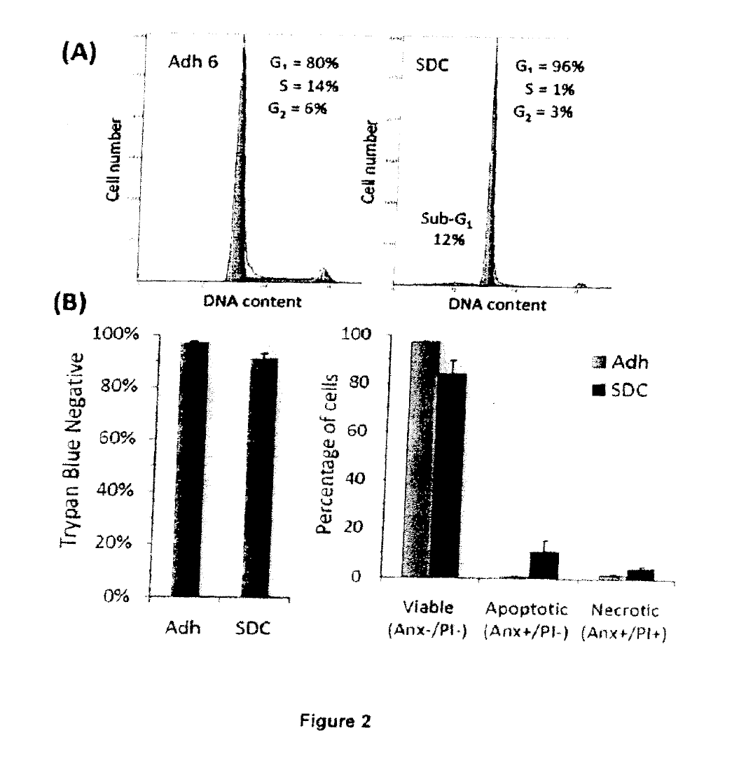

To compare proliferation rates and viability of adherent cells and spheroid-derived cells (SDCs), MSCs were plated as monolayers at 100 cells/cm² for 6 days (Adh 6), at 5,000 cells s/cm² for 3 days (Adh), and as hanging drops at 25,000 cells/drop for 3 days. Adherent MSCs were harvested with trypsin/EDTA. To obtain SDCs, spheroids were collected and dissociated with trypsin/EDTA. (FIG. 2A) For cell cycle analysis, MSCs were fixed with 70% Ethanol, washed in PBS, incubated with RNase, then stained with Prodium iodide (P1) overnight. DNA content was measured on a Beckman Coulter flow cytometer and data was analyzed using MultiCycle software (Phoenix Flow Systems). (FIG. 2B) The viability of MSCs derived from monolayers (Adh) and cultured spheroids (SDC) was determined with microscopy using trypan blue and by flow cytometry by labeling SDCs with Annexin V-FITC (Anx) and PI.

### MSC Spheroid-Derived Cells are Significantly Smaller than MSCs Cultured as Monolayers

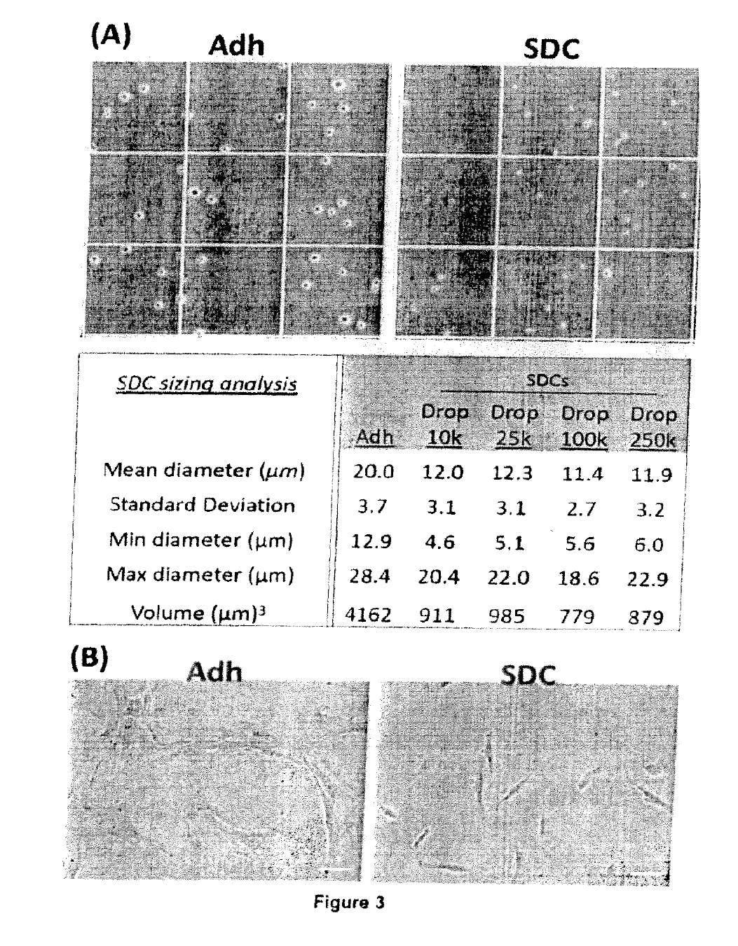

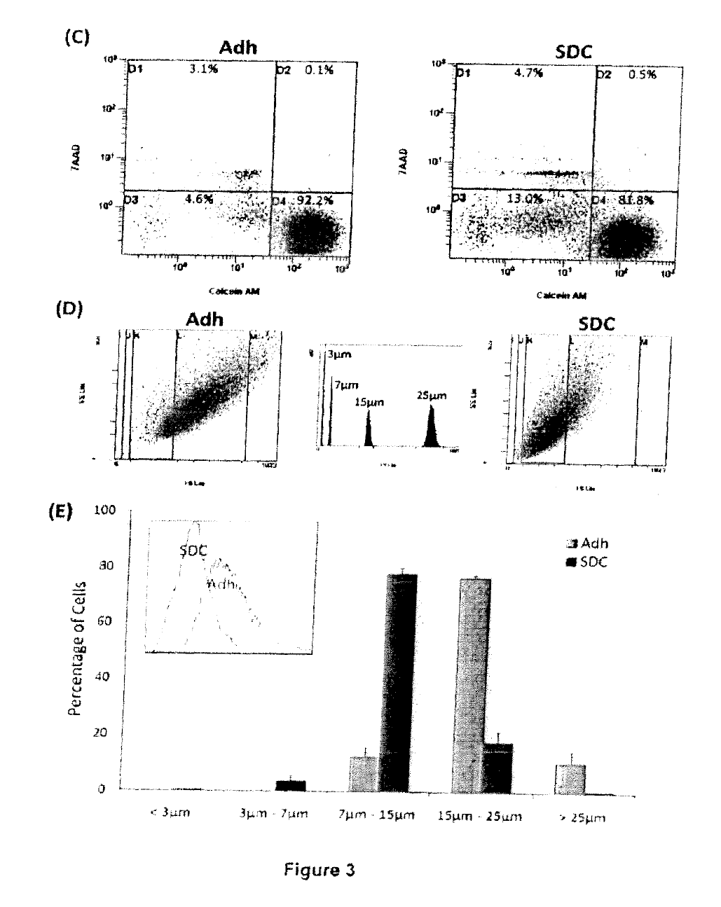

The size of MSCs cultivated for 3 days as monolayers and spheroids in hanging drops, was determined by microscopy and flow cytometry (FCM). (FIG. 3A) Cells derived from adherent monolayer cultures (Adh) and spheroids (SDCs) were transferred into chambers of a hemocytometer for analysis, Images were captured with a Nikon Eclipse Ti-S inverted microscope. Cell diameter (n>50 cells) was subsequently determined using NIS-Elements AR30 software and plotted as shown. SDC size was measured from cells acquired by the dissociation of spheres suspended in hanging drops for 3 days at varying cell densities; 10,000 (Drop-10k), 25,000 (Drop-25k), 100,000 (Drop-100k), 250,000 (Drop-250k). (FIG. 3B) Images captured 24 hours after plating Adh cells and SDCs further shows the smaller size of SDCs (Scale bar=50 μm). (FIG. 3C) 200,000 Adh cells and SDCs were incubated with the viability dyes calcein AM (live cells) and 7AAD (dead cells) for 10-20 min at RT before FCM. (FIG. 3D) Cell sizes were estimated from the viable population (Calcein+/7AAD−) by analyzing forward scatter (FS) properties using beads with known diameter (3, 7, 15, and 25 μm). Brackets were applied to the scatter plot at locations corresponding to the respective bead size as shown. (FIG. 3E) Gates established based on head size FS were used to group Adh and SDCs into five populations (<3 μm, 3-7 μm, 7-15 μm, 15-25 μm, and >25 μm).

### MSCs Cultured in Hanging Drops Display a Unique Expression Profile of Anti-Inflammatory, Anti-Apoptotic, Immune Modulatory, and Anti-Tumorigenic Genes

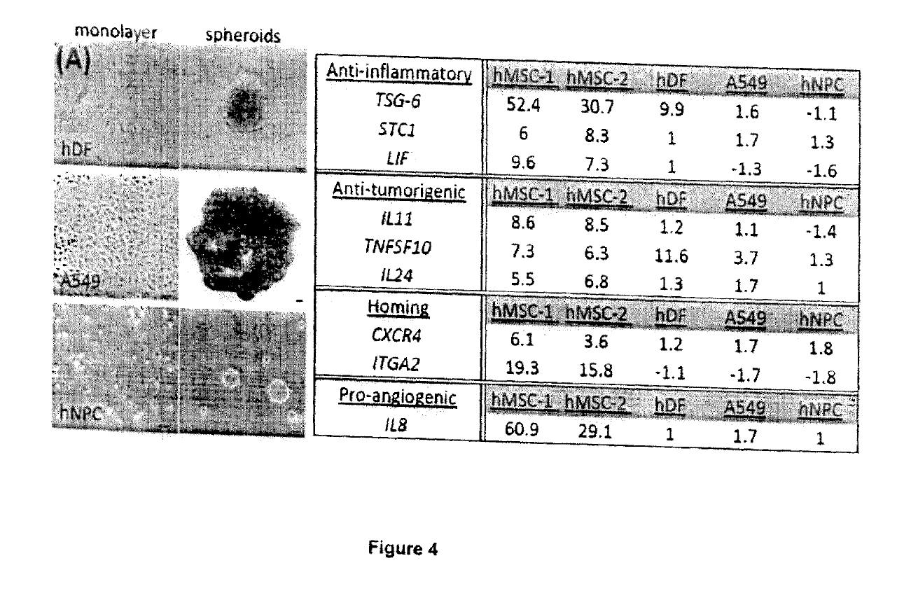

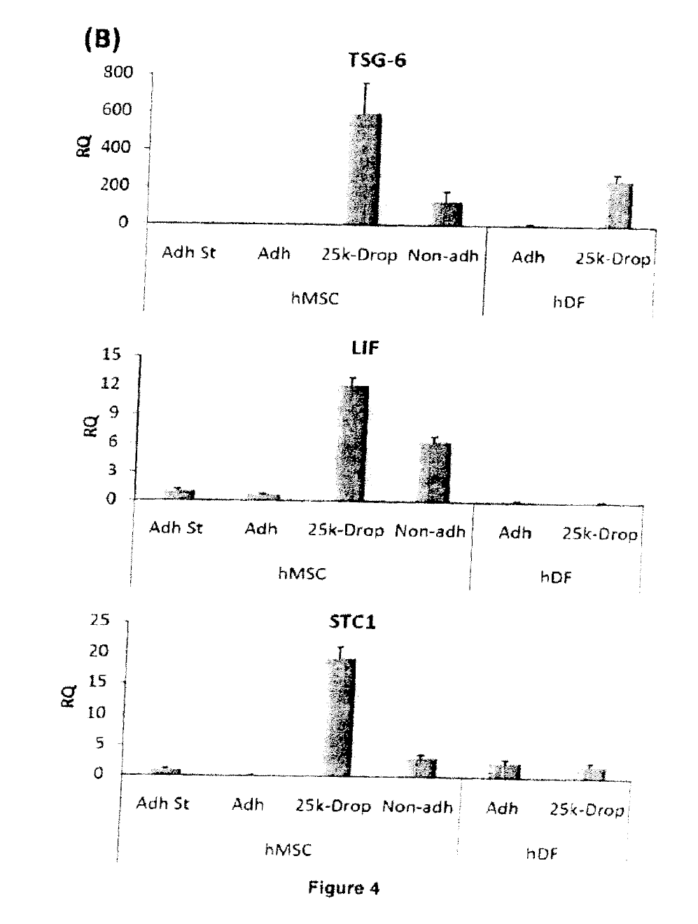

Human MSCs (MSC), human dermal fibroblasts (hDF), A549 lung carcinoma cells (A549), and human neural progenitors (hNPC) were suspended as hanging drops (25,000 cells/drop) to promote spheroid growth conditions. (FIG. 4A) After 3 days, the spheroids generated were harvested to isolate RNA for microarrays. Samples were hybridized on Human Exon 1.0 ST arrays and gene level analysis was performed with Partek Genomics Suite 6.4. Selected genes are displayed. The values shown represent fold changes compared to monolayer cultures of the respective cell type. (FIG. 4B) Microarray results of TSG-6, STC-1, and LIF were validated by real time RT-PCR in triplicate using 185 as an endogenous control. Results are shown as relative quantity (RQ) compared to adherent monolayer cultures (Adh). Scale bar=50 μm.

### Production of the Anti-Inflammatory Proteins TSG-6 and STC-1 is Enhanced Notably in MSCs Derived from Hanging Drop Cultures

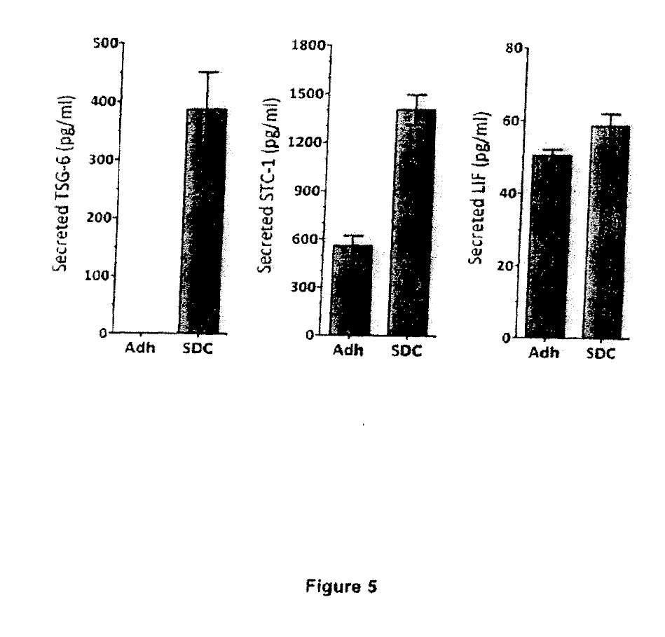

Multi-cellular spheroids, generated by suspending MSCs in hanging drops (25,000 cells/drop), were dissociated with trypsin/EDTA. The cells acquired (SDCs) were plated in 6-well plates at 200,000 cells/well in 1.5 ml of CCM. After 24 h, conditioned medium was harvested and utilized for ELISAs. The results obtained were compared to MSCs derived from adherent monolayer cultures (Adh) seeded at equal density/volume and also cultured for 24 h. Mean values are shown as secreted TSG-6, STC-1, or LIF (pg/ml). Error bars represent standard deviations. Experiments were performed in triplicate (FIG. 5).

### TNFα Levels Produced from LPS Stimulated Macrophages Were Decreased Markedly by MSC Spheroid-Derived Cells

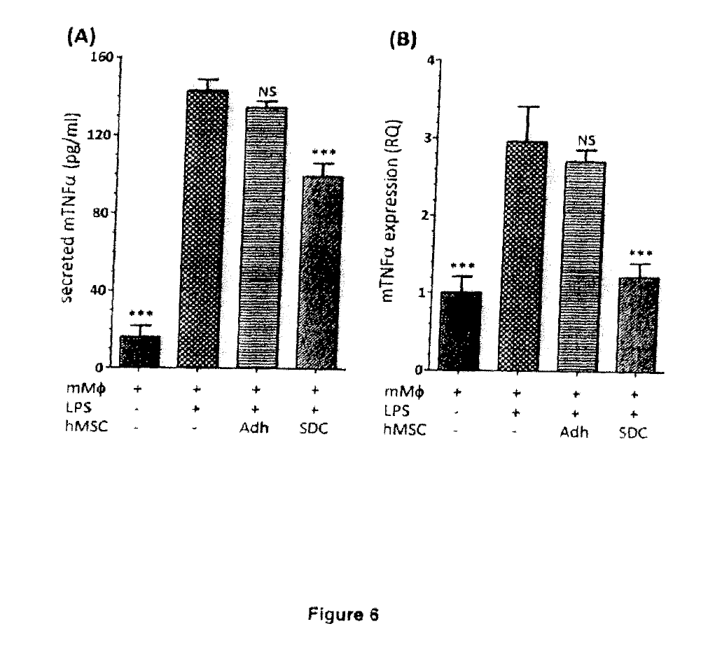

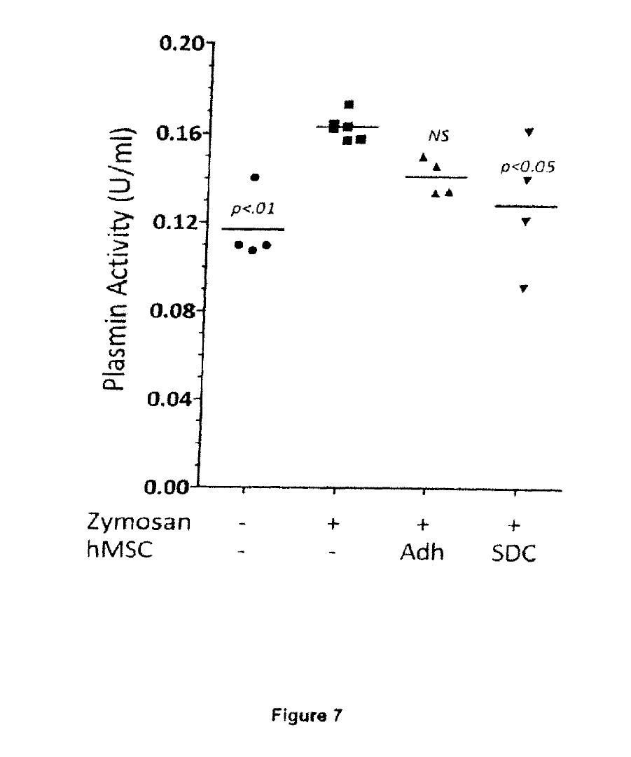

Mouse macrophages (mMcD), seeded in the upper chamber of a transwell (4.67 cm² growth area, 0.4 μm pores) at 400,000 cells/well, were stimulated with 0.1 μg/ml of LPS for 90 min. After LPS was removed, MSCs, derived from adherent monolayer cultures (Adh) and from spheroids generated by hanging drop technique (SDC), were plated beneath the transwell chamber at 200,000 cells/well. After 5 hours, medium conditioned by mMΦ was collected for mTNF-α ELISA (FIG. 6A). The cells then were harvested for RNA to quantify mTNF-α expression levels by RT-PCR (FIG. 6B). Values, expressed as mean±s.d. (n=3 per group), were subjected to ANOVA to evaluate levels of significance (***p<001).

### MSC Spheroid-Derived Cells Exhibit Anti-Inflammatory Effects in Vivo

To induce inflammation, C5713L/6 mice were injected IP with 1% zymosan. Fifteen minutes post injection, 1.5×10⁶ MSCs derived from adherent monolayer cultures (Adh) and hanging drops (SDC) were delivered into the peritoneal cavity. Blood was acquired from the right ventricle 24 hours later and allowed to clot to separate the serum. Serum plasmin activity, a marker of inflammatory status, then was ascertained by measuring time dependent cleavage of the substrate Chromozym PL into 4-nitraniline (absorbance=405 nm). Absorbance differences per minute subsequently were used to determine plasmin activity (U/ml). Each group consisted of 4-6 animals. Statistical significance was measured with ANOVA. (FIG. 6).

The results presented herein demonstrate the following: hMSCs suspended at high cell densities in hanging drops aggregate to form compact multi-cellular spheroids; mesenchymal surface features and growth characteristics are maintained and/or reacquired in the majority of hMSCs derived from multi-cellular spheroids; hMSCs cultured as multi-cellular spheroids in hanging drops show low proliferation rates but remain highly viable; hMSC spheroid-derived cells are significantly smaller than MSCs cultured on adherent dishes suggesting that SDCs may have enhanced mobility in the vasculature following infusion. In contrast to aggregates of human fibroblasts, cancer cells and neural progenitors, it was observed that hMSC spheroids exhibited a robust upregulation in expression of numerous anti-inflammatory (e.g., TSG-6, STC-1, LIF) and anti-tumorigenic (e.g., IL-11, TNFSF10, IL-24) genes. Significant upregulation of the homing receptor CXCR4 and the pro-angiogenic factor IL-8 also was exclusive to MSC spheroids. It was observed that hMSC spheroid-derived cells reduced TNF-α secreted by mouse macrophages and attenuated inflammatory response elicited by zymosan in a mouse model of peritonitis. Without wishing to be bound by any particular theory, the results presented herein demonstrate that hanging drop technique is useful in preprogramming MSCs to express therapeutically beneficial anti-inflammatory, anti-apoptotic, immune modulatory, and anti-tumorigenic proteins prior to transplantation.

### Example 2

### Aggregation of Human Mesenchymal Stromal Cells into Three Dimensional Multicellular Spheroids Enhances Their Anti-Inflammatory Properties

Recent studies showed that human mesenchymal stromal cells (hMSCs) trapped in the lungs of mice secreted an effective anti-inflammatory molecule, TSG-6, leading to improvements in the myocardial infarct model. Moreover, it has been shown that in hanging drop cultures, hMSCs aggregated into three dimensional spheroids and secreted proangiogenic factors. In the current work we sought to study in more detail the hMSC spheroid cultures, emphasizing their anti-inflammatory properties. hMSCs grown in hanging drops or on low adherent dishes aggregated rapidly, and formed spheroids with increased TSG-6 expression. After 72-96 hours in hanging drops the expression or TSG-6 peaked, more than 1000-fold over their monolayer counterparts, but the viability of the cells derived from spheroids declined significantly after 72 hours. Microarray analysis of spheroid and monolayer cultures demonstrated a high expression of several anti-inflammatory and cell adhesion molecules in spheroids, while the expression of cell-cycle genes was down regulated. The expression of TSG-6, LIF, and STC-1, in spheroids, was confirmed with real-time RT-PCR. The production of these anti-inflammatory proteins per cell was enhanced markedly in spheroids relative to monolayer cultures as demonstrated with protein specific ELISA. Furthermore, we showed that hMSC spheroids, but not monolayer hMSCs, significantly suppressed the mTNF-α secretion by LPS stimulated macrophages in a co-culture system, verifying the anti-inflammatory capacity of the spheroids. Moreover, hMSC spheroids were anti-inflammatory in a mouse model of peritonitis. Overall, we demonstrated that hMSCs can he activated to secrete high amounts of anti-inflammatory cytokines, without chemical induction, by culturing them in hanging drops or on low adherent dishes as spheroids. The results suggested that hMSC spheroids could be used as anti-inflammatory therapy in many diseases.

### hMSCs Aggregated Rapidly into Three Dimensional Spheroids When Grown in Hanging Drops or on a Nonadherent Surface

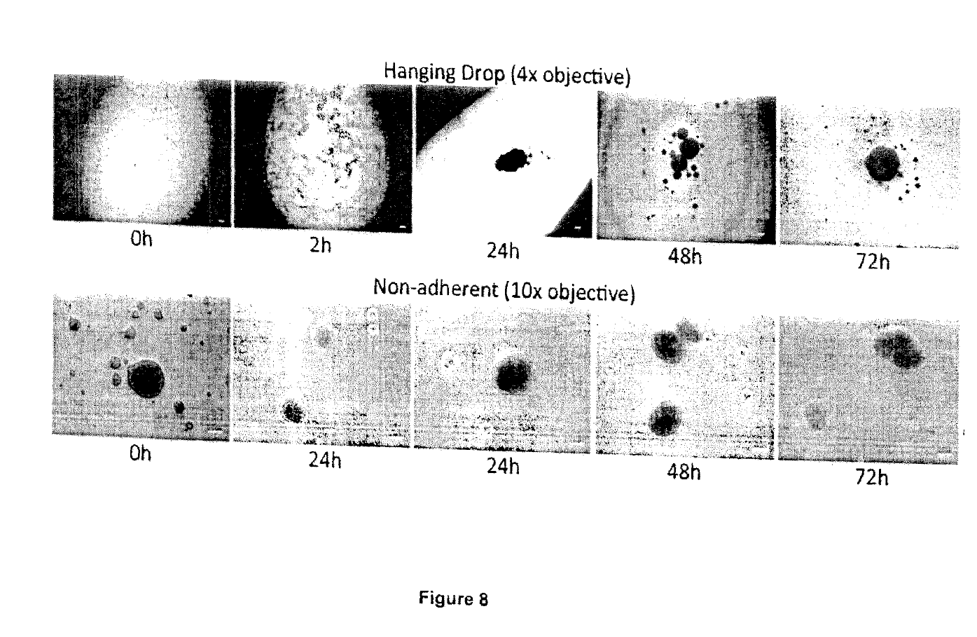

Human MSCs were isolated from 1-4 ml bone marrow aspirates taken from the iliac crest of normal adult donors. Nucleated cells were isolated with density gradient and resuspended in complete culture medium (CCM): α-MEM, 17% FBS, 100 units/ml penicillin, 100 μg/ml streptomycin, and 2 mM L-glutamine. Nucleated cells were plated on a 175 cm² culture flask and incubated at 37° C. with 5% CO₂. After 24 hours, nonadherent cells were discarded, Adherent cells were incubated 4-11 days until approximately 70% confluent, harvested with 0.25% trypsin and 1 mM EDTA for 5 mm at 37° C., and replated at 50 cells/cm² in an intercommunicating system of culture flasks. The cells were incubated 7-12 days until approximately 70% confluent, harvested with trypsin/EDTA, and frozen in 5% DMSO and 30% FBS as passage 1 cells. A frozen vial of passage 1 MSCs (donor 1 or 2) was thawed and the cells were plated in a 145 cm² culture dish in CCM. After 24 hours of incubation, adherent cells were harvested with trypsin/EDTA, plated at 100 cells/cm², and expanded for 7 days before freezing. In this study, passage 1 or 2 frozen MSCs were recovered, harvested and grown at 100 cells/cm² for 7-8 days before collecting for various assays. To generate spheroids for images, MSCs were plated either as hanging drops on the lid of a culture dish in 35 μl of CCM (25,000 cells/drop), or on non-adherent surface at 200,000 cells/ml for up to 3 days, Scale bar 100 μm. It was observed that hMSCs aggregated rapidly into three dimensional spheroids when grown in hanging drops or on a nonadherent surface (FIG. 8).

### hMSCs in Smaller Spheroids Show High Viability

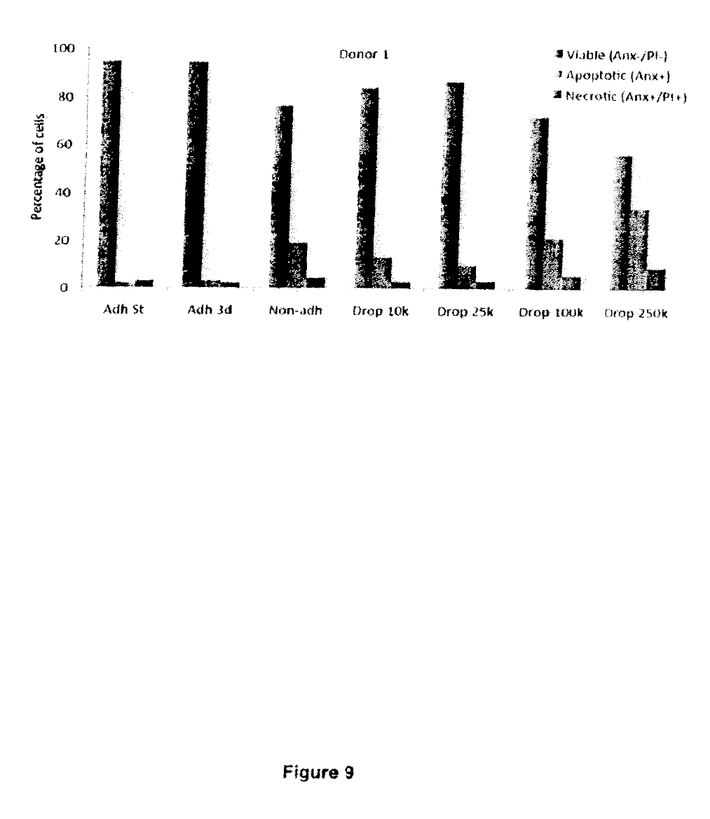

MSCs from 2 donors were plated at 100 cells/cm² and grown as monolayers for 7-8 days before harvesting (Adh-St) for viability assay by FACS using Annexin (Anx) and Propidium iodide (P1) staining. Harvested cells were also plated on adherent dishes at 5000 cells/cm² (Adh), on nonadherent dishes at 200,000 cells/ml (Non-adh), and as hanging drops (Drop) at different cell densities; 10,000 (10k), 25,000 (25k), 100,000 (100k), and 250,000 (250k), Cells from all conditions were harvested for viability assay at day 3. Viability assay also was performed on cells from monolayer (Adh) and 25k spheroid cultures (25k Drop) at day 1 (1 d), day 2 (2 d), day 3 (3 d), and day 4 (4 d). ft was observed that hMSCs in smaller spheroids show high viability (FIG. 9).

### hMSC Spheroids Express High Levels of Anti-Inflammatory Molecule TSC-6

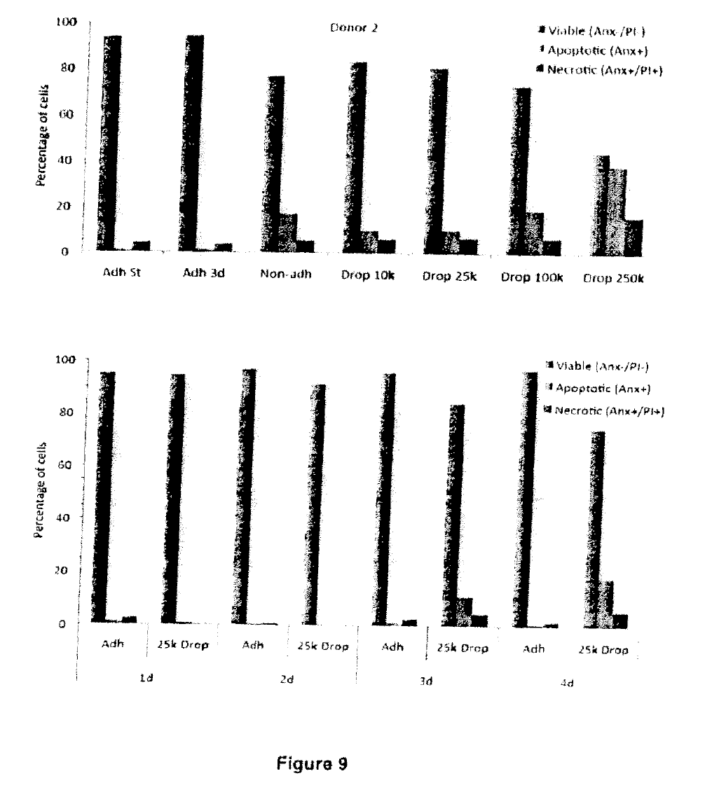

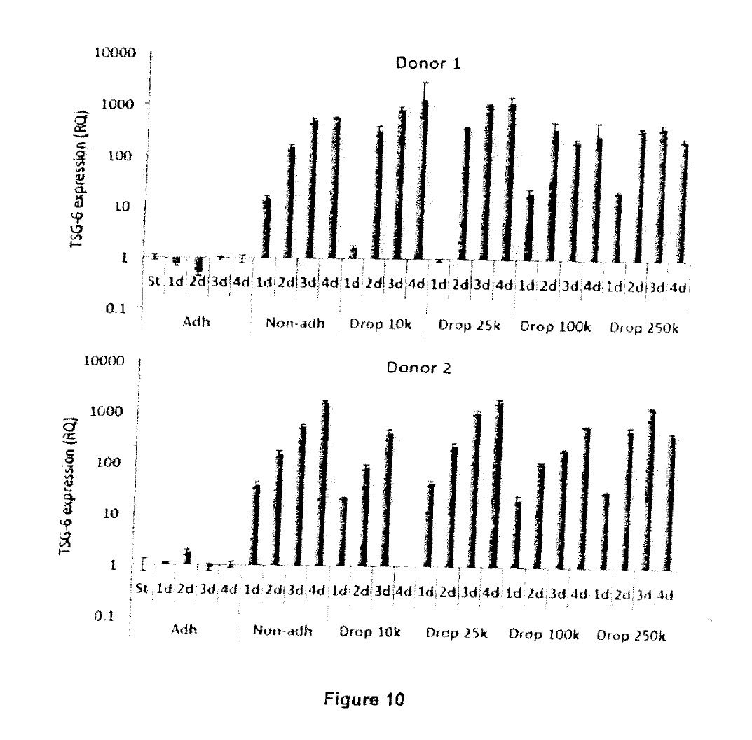

MSCs from 2 donors were plated at 100 cells/cm² and grown as monolayers for 7-8 days before harvesting for RNA (Adh-St) and subsequent cultures. Harvested cells were plated on adherent dishes at 5000 cells/cm² (Adh), on non-adherent dishes at 200,000 (Non-adh), and as hanging drops (Drop) at different cell densities; 10,000 (10k), 25,000 (25k), 100,000 (100k), and 250,000 (250k). Cells were harvested for RNA at day 1 (1 d), day 2 (2 d), day 3 (3 d), and day 4 (d4). Real-time RT-PCR for TSG-6 was performed using TaqMan Gene Expression Assay with 18S as an endogenous control in triplicate. Results are shown as relative quantity (RQ) compared to Adh-St sample (FIG. 10). Error bars are 95% confidence intervals.

### hMSC Spheroids Express High levels of Several Cytokines and Cell Adhesion Molecules While the Expression of Cell Cycle and Cytoskeletal Genes is Down-Regulated

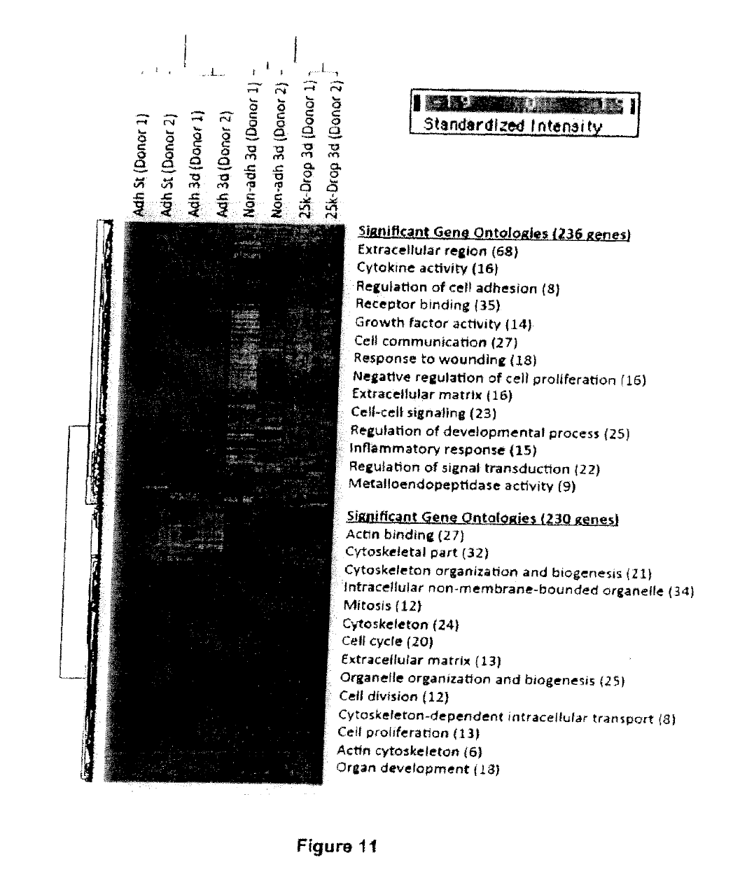

MSCs from 2 donors were plated at 100 cells/cm² and grown as monolayers for 7 days before harvesting for RNA (Adh-St) and subsequent cultures. Harvested cells were plated on adherent dishes at 5000 cells/cm² (Adh), on non-adherent dishes at 200,000 cells/ml (Non-adh), and as hanging drops at 25,000 cells/drop (25k-Drop). Cells were harvested for RNA at day 3 (3 d) and prepared for microarray using Whole Transcript Sense Target Labeling Assay. Labeled and fragmented samples were hybridized on Human Exon 1.0 ST arrays and gene level analysis was performed with Partek Genomics Suite 6.4. Genes that were either up- or down-regulated in spheroids (25k-Drop 3 d) at least 2-fold compared to their monolayer counterparts (Adh-St and Adh-3 d), were used in hierarchical clustering. The most significant Gene Ontology terms for up- and down-regulated genes are shown next to the heat map (FIG. 11).

### hMSC Spheroids Express High Levels of Anti-Inflammatory and Anti-Tumorigenic Molecules

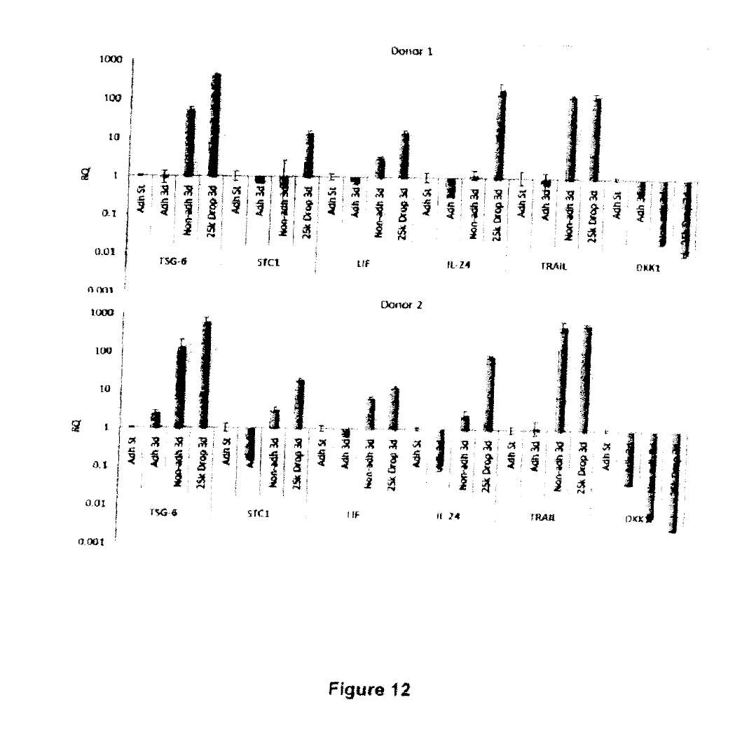

Microarray results were confirmed with real-time RT-PCR for several anti-inflammatory (TSG-6, STC-1, and LIF) and anti-tumorigenic (IL-24 and TRAIL) molecules, and a Wnt signaling inhibitor (DKK1); FIG. 12. Results are shown as relative quantity (RQ) compared to MSCs plated at 100 cells/cm² on adherent dishes and grown for 7 days (Adh St). MSCs were grown for 3 days on adherent dishes at 5000 cells/cm² (Adh 3 d), on non-adherent dishes at 200,000 cells/ml (Non-adh 3 d), and as hanging drops at 25,000 cells/drop (25k-Drop 3d). 18S was used as an endogenous control. Error bars are 95% confidence intervals from triplicate reactions.

### hMSC Spheroids Secrete Large Amounts of Anti-Inflammatory Cytokines

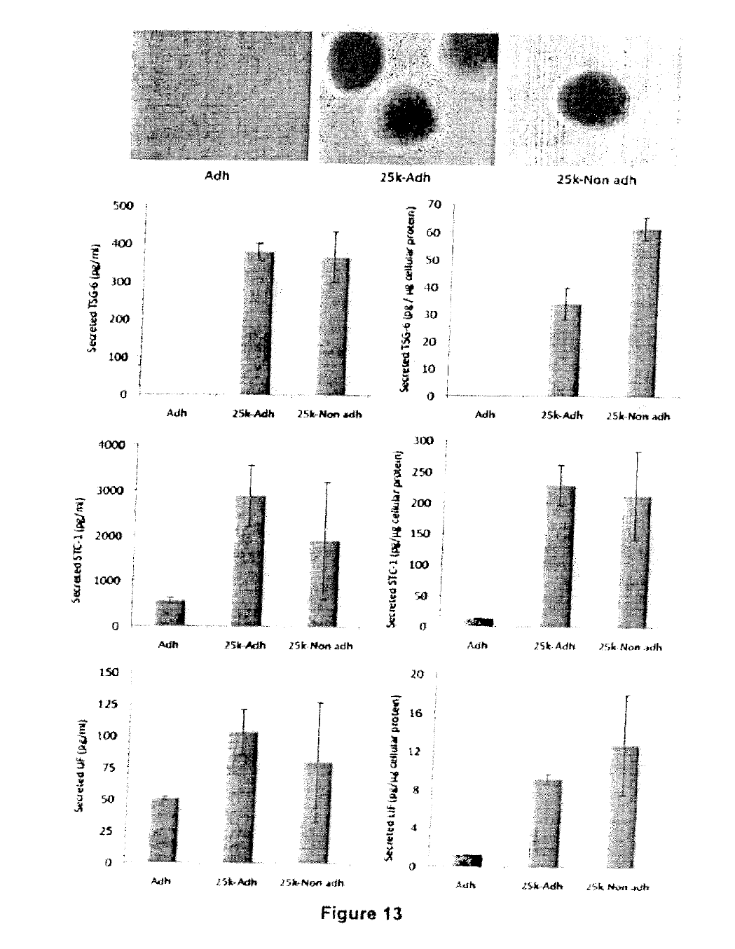

MSCs were plated at 100 cells/cm² and grown as monolayers for 7 days. Cells were harvested and plated at 5000 cells/cm² on adherent dishes and as hanging drops at 25,000 cells/drop. After 3 days, monolayer cultures were harvested and plated on adherent dishes (Adh) at 200,000 cells/well in 1.5 ml of CCM in triplicate. Spheroids grown for 3 days were plated either on adherent (25k-Adh) or non-adherent (25k-Non adh) dishes at 8 spheroids/well in 1.5 ml of CCM in triplicate. Conditioned medium was collected after 24 hours, cleared from cellular material, and used for ELISAs. Cells were lysed for total cellular protein measurements to account for the loss of cells during transfer. Values are shown as secreted TSG-6, STC-1, or LIF (μg/ml) or as secreted TSG-6, STC-1, or LIF (μg) per cellular protein (μg) after subtraction of CCM signal (FIG. 13). Error bars are standard deviations from triplicate experiments.

### LPS Stimulated Macrophage Secrete Less TNF-α When Co-Cultured with hMSC Spheroids

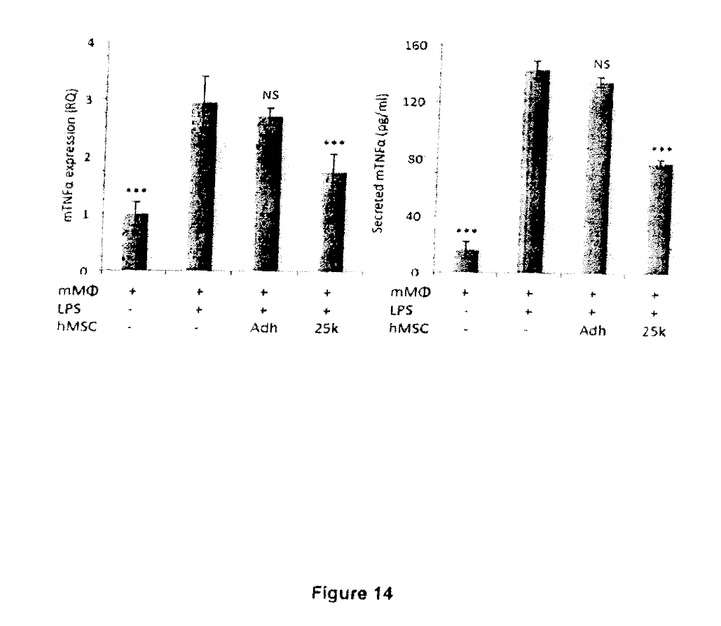

Mouse macrophages (mMΦ) were plated in the upper chamber of the transwell (0.4 μm) at 400,000 cells/well. Cells were stimulated with 0.1 μg/ml of EPS in DMEM supplemented with 10% FBS and penicillin/streptomycin. After 90 min, LPS was removed and replaced with fresh medium and MSCs were plated in the bottom chamber of the transwell at 200,000 cells/well (Adh), or 8 spheres/well (25k). After 5 hours, mMΦ were harvested for RNA and subsequent real-time RT-PCR for mTNF-α. In addition, conditioned medium was harvested for TNF-α ELISA (FIG. 14). Error bars are standard deviations from triplicate experiments. ANOVA was used to determine the significance levels.

### hMSC Spheroids Show Anti-Inflammatory Effects in a Mouse Model of Peritonitis

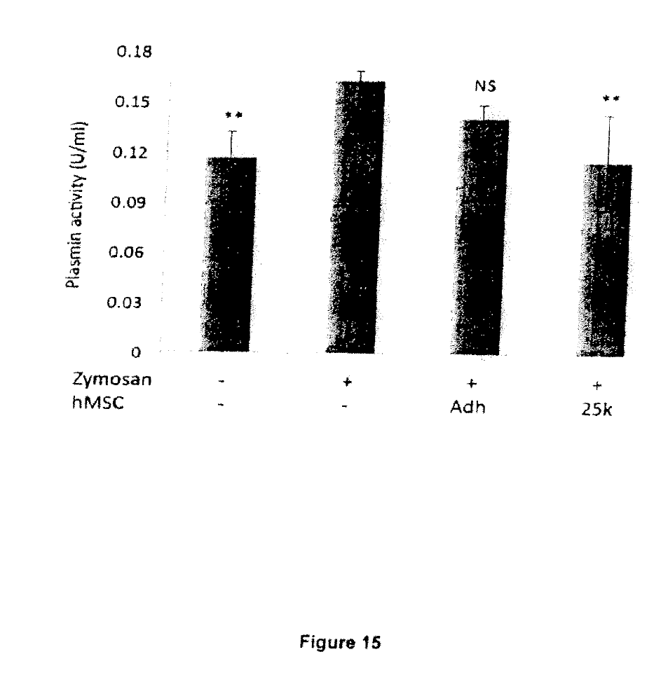

C57BL/6 mice were injected IP with 1% zymosan in 1 ml of HBSS. Total of 1.5×10⁶ monolayer MSCs (Adh) or 60 spheres (25k) were injected IP 15 min. later in 160 μl of HBSS. After 24 hours, blood was collected from the right ventricle and serum was isolated. Plasmin activity was measured from the serum with Chromozym PL cleavage reaction (FIG. 15). Error bars are standard deviations for 4-6 animals per group. ANOVA was used to determine the significance levels.

The results presented herein demonstrate the following: hMSCs aggregate rapidly into spheroids when grown in hanging drops or on non-adherent dishes; hMSC spheroids show high viability and TSG-6 expression when grown as small spheroids (25k) for 3 days; hMSC spheroids express high levels of anti-inflammatory (TSG-6, STC-1, and LIF) and anti-tumorigenic (IL-24 and TRAIL) molecules and secrete high levels of the anti-inflammatory proteins; hMSC spheroids show anti-inflammatory effects in an in vitro inflammation assay and are anti-inflammatory in a mouse model of peritonitis. The results suggest that hMSC spheroids are useful as an anti-inflammatory therapy in many diseases.

### Example 3

### Materials and Methods

hMSC Cell Culture. Frozen vials of passage 1 hMSCs from bone marrow were obtained from the Center for the Preparation and Distribution of Adult Stem Cells (http://Medicine.tamhsc.edu/irm/msc-distribution.html). After 24-hours recovery, hMSCs were seeded at low density (100 cells/cm²), and incubated in complete culture medium (CCM) containing 17% FBS for 7-8 days until approximately 70% confluent. hMSCs were passed under the same conditions through no more than three passages before being used for assays.

Spheroid Generation and Dissociation. hMSCs were plated in hanging drops in 35 μl of CCM containing 10,000-250,000 cells/drop for up to 4 days. To obtain spheroid derived cells, spheroids were incubated with trypsiniEDTA for 5-30 min (depending on the size of the spheroid) while pipetting every 2-3 min.

Intravenous Infusion of hMSCs and Mu PCR. Male NOD/scid mice were infused with 106 monolayer or spheroid derived hMSCs i.v. followed by collection of tissues 15 min later. Genomic DNA was isolated and used to determine the relative quantity of human DNA in each tissue with real-time PCR for human Alu and GAPDH and mouse GAPDH. (Lee, et al., Cell Stem Cell, Vol. 5, pgs. 54-63 (2009); Lee, et al., Blood, Vol. 113, ps. 816-826 (2009); McBride, et al., Cytotherapy, Vol. 5, pgs 7-18 (2003)).

Mouse Model of Peritonitis and Measurements of Inflammation. To induce inflammation in male C578L/6J mice, zymosan solution was administered i.p., followed by i.p. injection of either 1.5×10⁶ monolayer hMSCs, 1.5×10⁶ spheroid derived cells, or 60 spheroids 15 min later, After 6 hours, inflammatory exudates were collected by peritoneal lavage and the cell-free supernatant was used to measure total protein, neutrophil activity (secreted mMPO), and levels of the pro-inflammatory molecules mTNFα, mIL-1β, mCXCL2/MIP-2, and PGE2. Twenty-four hours after cell injection, blood was collected from the right ventricle and the mouse plasmin activity was measured from the serum.

### Results

Aggregation of hMSCs in Hanging Drops into Spheroids. To aggregate hMSCs, we used a hanging drop protocol. Time-lapse microscopy demonstrated that hMSCs cultured in hanging drops first formed a loose network and then numerous small aggregates that gradually coalesced into a single central spheroid along the lower surface of the drop (FIG. 16A). Once assembled, the spheroid did not increase in size but compacted progressively between 48 and 96 hours. H&E staining of sections revealed the spheroids were solid throughout with small round cells evenly distributed and embedded in matrix (FIG. 16B). The surface of the spheroid had a layer of epithelium-like cells that were more elongated and flatter. As expected, the sizes of the spheroids were dependent on the number of hMSCs suspended in the hanging drops (FIG. 16E). hMSC spheroids of all sizes expressed and secreted very high levels of the anti-inflammatory molecule TSG-6 compared with either low or high density monolayer cultures, but spheroids of 25,000 cells (Sph 25k) showed the highest expression and secretion of TSG-6 (FIGS. 16C and D). Moreover, TSG-6 expression increased in a time dependent manner with spheroids of 25,000 hMSCs and consistently was much higher than in standard cultures of adherent hMSCs (FIG. 16F).

Viability of hMSCs in Spheroids. Because hMSCs in spheroids may have less access to nutrients, it was of interest to establish whether the cells remained viable. In 3 day cultures of spheroids of 10,000 or 25,000 hMSCs, almost 90% of the harvested cells were viable as assayed by propidium iodide (PI) uptake and labeling with annexin V-FITC (FIG. 17A). The number of apoptotic or necrotic cells was greater in spheroids prepared with 100,000 or 250,000 hMSCs (FIG. 17A). Also, the number of apoptotic or necrotic cells increased slightly when the incubation period was extended from 3 days to 4 days (FIG. 17B).

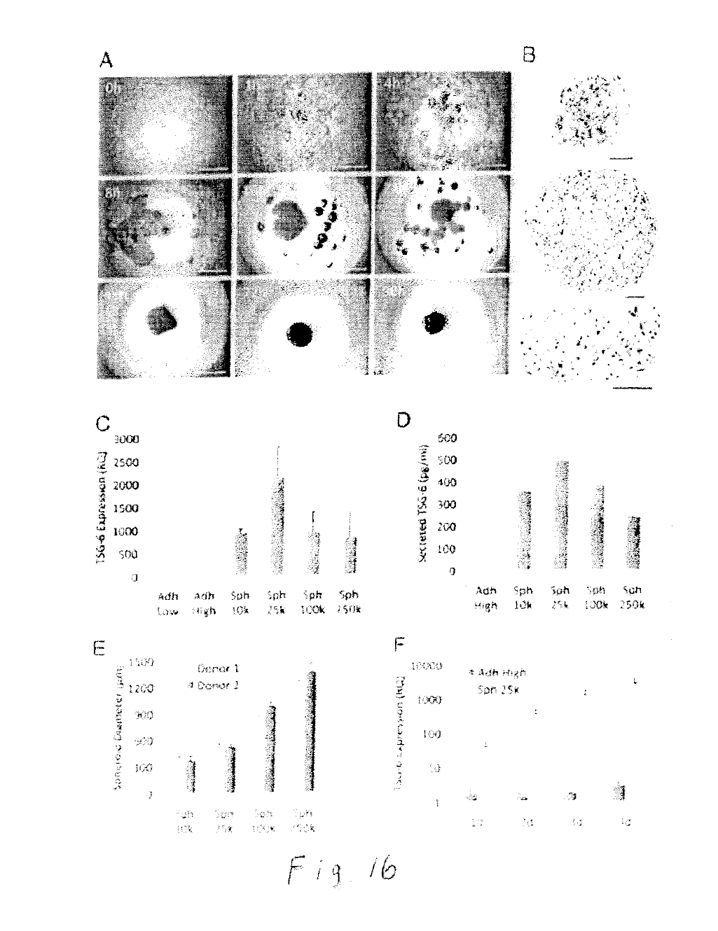

Analysis of Spheroid hMSC Size in Vitro and Relative Tissue Distribution After i.v. Infusion. As suggested by histological sections (FIG. 6B), hMSCs in spheroids appeared smaller than hMSCs from standard monolayer cultures. The cells released from spheroids by tripsinization were nearly half the diameter and approximately one-fourth the volume of hMSCs derived from adherent monolayers as shown by flow cytometry (FIG. 18A) and microscopy (FIG. 18B).

In order to test if the smaller size of the hMSCs dissociated from spheroids would allow the cells to traffic through the lung micromusculature and therefore distribute more efficiently into other tissues, both monolayer and spheroid hMSCs were injected i.v. into the tail vein of NOD/scid mice. Real-time PCR for human Alu sequences in the lungs collected 15 min after hMSC infusion suggested that the number of trapped cells decreased by about 25% with spheroid-derived hMSCs compared with monolayer hMSCs. At the same time, a larger fraction of infused spheroid hMSCs were recovered in the liver, spleen, kidney, and heart (FIG. 18C).

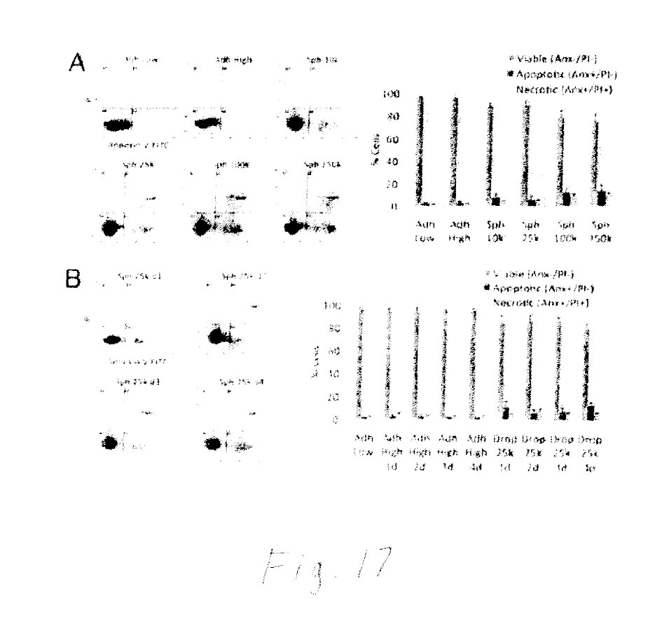

hMSCs Dissociated from Spheroids Retain the Properties of Adherent hMSCs. hMSCs dissociated from spheroids retained the ability to differentiate into mineralizing cells and adipocytes (FIGS. 19A and B). The dissociated cells expanded more slowly during an initial passage and then more rapidly than adherent hMSCs through four passages before reaching senescence at about the same number of population doublings (FIG. 19C). In addition, the dissociated cells readily generated colonies (CFUs) when plated at clonal densities (FIG. 19D). Consistent with the data on rates of propagation (FIG. 19C), the number of CFUs from spheroid cells was initially less than the number of CFUs from adherent cultures but was greater in later passages (FIG. 19D). The surface epitopes of the hMSCs dissociated from spheroids were similar to the surface epitopes of hMSCs from adherent monolayers when dissociated under the same conditions with trypsin (10 mm at 37° C.): the dissociated cells were negative for hematopoietic markers, and they were slightly less positive for CD73, CD90, and CD105, apparently because of the smaller size of the cells (FIG. 19F).

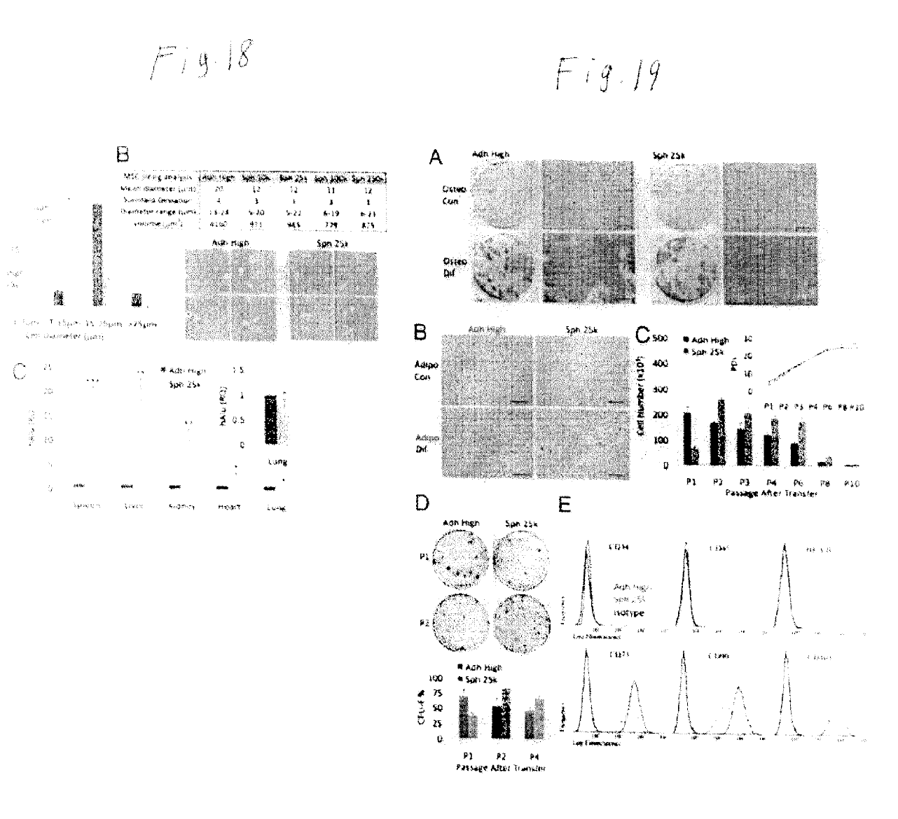

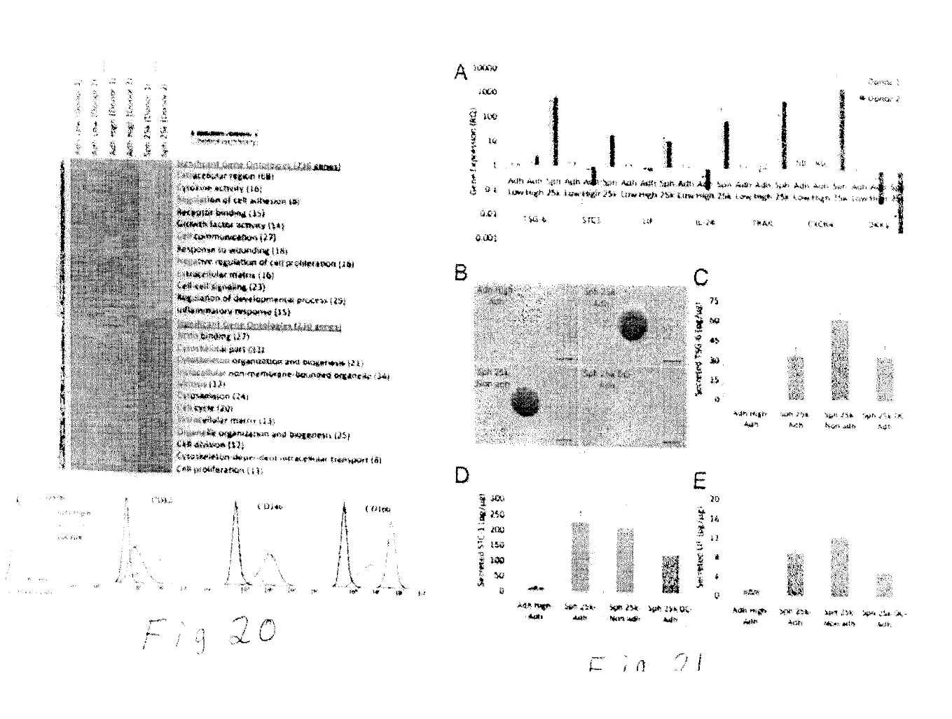

Transcriptome Changes in the Spheroid hMSCs. Surveys with microarray assays demonstrated that 236 genes were up-regulated and 230 genes were down-regulated in a comparison of spheroid cells with hMSCs from adherent monolayers (FIG. 20A). There were increases in genes with ontologies for the extracellular region, regulation of cell adhesion, receptor binding, cell communication, extracellular matrix, and negative regulation of cell proliferation (FIG. 20A). Also, there were parallel decreases in genes with ontologies for cytoskeleton organization and biogenesis, mitosis, cell cycle, and extracellular matrix (FIG. 20A). Of special interest was the increase in genes with ontologies for response to wounding and inflammatory response (FIG. 20A). Real time RT PCR assays (FIG. 21A) demonstrated marked increases in the expression of TSG-6; stanniocalcin-1 (STC-1), an anti-inflammatory/anti-apoptotic protein; leukemia inhibitory factor (LIF), a cytokine for growth and development; IL-24, a tumor suppressor protein; TNF-α related apoptosis inducing ligand (TRAIL), a protein with selectivity for killing certain cancer cells; and CXC chemokine receptor 4 (CXCR4), a receptor involved in MSC homing. As expected from its stimulatory effect of MSC proliferation (Gregory, et al., J. Biol. Chem., Vol 278, pgs. 28067-28078 (2003)), there was decreased expression of dickkopf 1 (DKK1), an inhibitor of Wnt signaling (FIG. 21A).

Changes in Cell Surface Protein Expression and Cell Cycle Distribution in hMSC Spheroids. Assays by flow cytometry demonstrated decreased expression of podocalyxin-like protein (PODXL), an anticell-adhesion protein; and α4-integrin (CD49d), an integrin subunit associated with lymphocyte homing. There was partial down-regulation of the melanoma cell adhesion molecule (MCAM or CD 146) that is used as a marker for endothelial cells and pericytes, and of ALCAM (CD 166), a cell adhesion molecule (FIG. 20B). At the same time, there was increased expression of an integrin subunit for cell adhesion (α2-integrin of CD49b), and a protein associated with suppression of metastases (CD82) (FIG. 20B). As expected from microarray results, assays by flow cytometry also demonstrated a decrease of spheroid hMSCs in S-phase compared with monolayer hMSCs.

Spheroid hMSCs Secrete Anti-inflammatory Proteins. Spheroids of hMSCs plated on adherent culture surfaces gradually generated spindle-shaped cells that migrated away from the spheroids (FIG. 21B). No migration was seen with spheroids plated on non-adherent surfaces (FIG. 21B). ELISAs demonstrated that hMSCs either in spheroids or dissociated from spheroids continued to secrete TSG-6, STC-1, and LW when plated on culture dishes for 24 hours (FIG. 21C-E). The levels of all three factors were much higher than with adherent monolayer hMSCs. About the same levels of STC-1 and LIF were observed in spheroids cultured directly either on adherent or non-adherent plates, but spheroids cultured on non-adherent dishes secreted more TSG-6 (FIG. 21C-E). The levels of TSG-6, STC-1, and LIF decreased when the hMSCs were dissociated from spheroids and cultured on adherent plates but the levels remained much higher than with adherent monolayers (FIG. 21C-F).

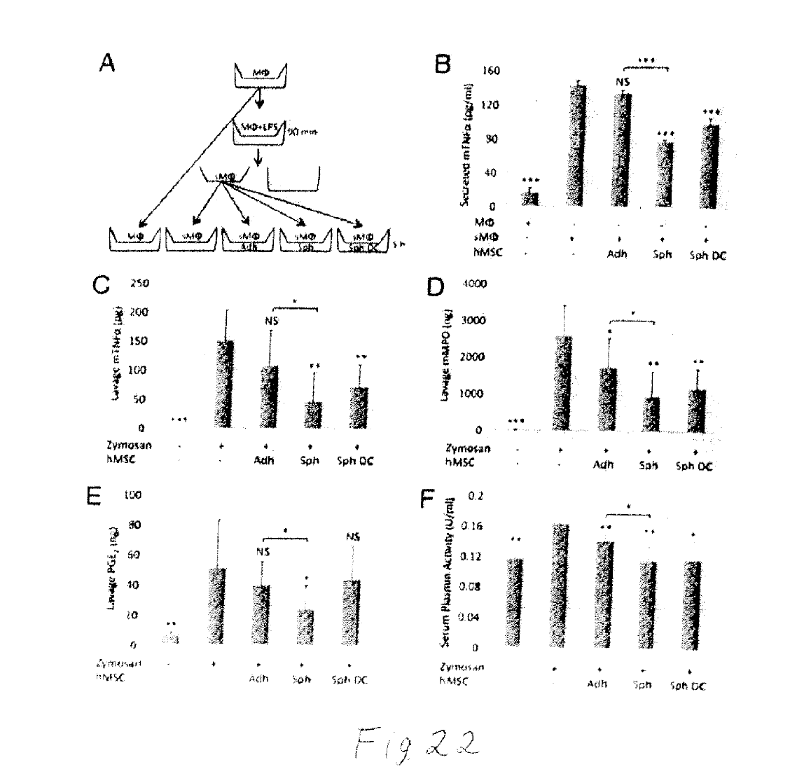

Spheroid hMSCs Decrease Activation of Macrophages in Vitro and Inflammation in Vivo. The increased secretion of anti-inflammatory molecules TSG-6 and STC-1 by the spheroid hMSCs suggested that the cells would he more effective than adherent monolayer cultures of hMSCs in reducing inflammatory responses. To test this prediction, mouse macrophages were pre-activated with LPS in the upper chamber of a transwell, followed by a transfer of the chamber to a test well (FIG. 22A). Under the conditions of the experiment, the presence in the test well of hMSCs from adherent monolayers had no significant effect on the expression or secretion of TNFα by the stimulated macrophages (FIG. 22B). In contrast, TNFα expression and secretion was decreased significantly by the presence in the test well of intact spheroids or hMSCs dissociated from spheroids (FIG. 22B). The results indicated therefore that the spheroid derived hMSCs secreted more effective anti-inflammatory factors.

In addition, the increased expression of STC-1 is important because STC-1 also reduces reactive oxygen species, or ROS. ROS are an early trigger for inflammation, and apoptotic at high levels. Thus, STC-1 is anti-inflammatory and anti-apoptotic.

To test the effects of spheroid hMSCs on inflammation in vivo, a mouse model of zymosan-induced peritonitis was used (Schwab, et al., Nature, Vol. 447, pgs. 869-874 (2007)). Six hours after i.p. administration of monolayer, spheroid, or spheroid derived hMSCs, inflammatory exudates were collected and used in estimating the level of inflammation. hMSC spheroids significantly decreased the protein content of the lavage fluid and the volume, neutrophil activity, as assayed by secreted myeloperoxidase (MPO) (FIG. 22D), and levels of the pro-inflammatory molecules TNFα (FIG. 22C), IL-1β, CXCL2/MIP-2, and PGE₂ (FIG. 22E). In addition, serum levels of plasmin activity, an inflammation associated protease that is inhibited by TSG-6 (Wisniewski, et al., Cytokine Growth Factor, Vol. 15, pgs. 129-146 (2004)), were decreased significantly by hMSC spheroids (FIG. 22F). Serum plasmin activity was reduced approximately to the levels of non-inflammatory control animals 24 hours after spheroid injection (FIG. 22F). Spheroid-derived hMSCs also substantially decreased levels of the inflammatory markers assayed, although to a lesser extent than intact spheroids (FIG. 22 C-F). Moreover, hMSC spheroids were significantly more effective than adherent monolayer hMSC in suppressing inflammation (FIG. 22 C-F).

### Discussion

Classically hMSCs were isolated and expanded as adherent monolayer cultures, but it was soon recognized that centrifugation of the cells to form micropellets or large aggregates greatly enhanced their chondrogenic differentiation that slowly occurred over several weeks (Arufe, et al,. J. Cell Biochem., Vol. 148, pgs 145-155 (2009); Johnstone, et al., Exp. Cell, Res., Vol. 238. pgs. 265-272 (1998)). However, several recent publications demonstrated that culture of MSCs in 3D or as spheroids for shorter periods of time improved their therapeutic potential by increased expression of genes such as CXCR4 to promote adhesion to endothelial cells or of IL-24 that has tumor suppressing properties (Potopova, et al., J. Biol. Chem., Vol. 283, pgs. 13100-13107 (2008): Frith, et al., Tissue Eng. Part C Methods, Vol, 16, No. 4, pgs 735-749 (2010); Wang et al., Stem Cells, Vol. 27, pgs. 724-732 (2009)). The experiments presented here were designed to prepare hMSCs as spheroids that maximally expressed TSG-6, the anti-inflammatory protein that produced beneficial effects in mice with myocardial infarcts because it was expressed at high levels after i.v.-infused hMSCs were trapped in the lung (Lee, Cell Stem Cell, 2009).

The results demonstrated that the properties of hMSCs cultured as spheroids depend critically on the experimental conditions. In hanging drops, the cells first formed a network and then most of the cells coalesced into a single spheroid. Optimal levels of TSG-6 expression were observed with spheroids approximately 500 μm in diameter and incubated for 3 days. Expression levels remained high but were lower in larger spheroids, and more of the cells became apoptotic or necrotic in the larger spheroids. Also, more of the cells became apoptotic or necrotic with longer times of incubation. The cells in spheroids retained most of the surface epitopes of hMSCs from adherent cultures, Also, hMSCs dissociated from spheroids retained the potential to differentiate into mineralizing cells and adipocytes. They also expanded at a similar rate as hMSCs from adherent monolayer cultures after a delay through one passage. In addition, spheroid-dissociated hMSCs remained highly clonogenic.

As was observed previously with large hMSC spheroids (Potopova, et al., Stem Cells, Vol. 25, pgs. 1761-1768 (2007)) and hMSCs in 3D culture (Frith, 2010), surveys with mRNA/cDNA microarrays demonstrated marked differences in the transcriptomes compared with hMSCs from adherent cultures. Quantitative assays confirmed some of the important differences. As expected, there was a marked decrease in the anticell-adhesion protein PODXL (Lee, Blood, 2009) and a decrease in cell cycling. Of special note was that several of the differences had important implications for the potential therapeutic uses of hMSCs. There were higher levels of expression of the anti-inflammatory protein TSG-6 than previously observed by pre-incubation of hMSCs with TNFα (Lee, Cell Stem Cell 2009). Also, there was a high level of expression of STC-1, a protein with both anti-inflammatory and anti-apoptotic effects (Block et al., Stem Cells, Vol. 27, pgs. 670-681 (2009); Huang, et al., Am. J. Pathol. Vol. 174. pgs. 1368-1378 (2009)). The high levels of expression of both TSG-6 and STC-1 were maintained for at least 1 day after the cells were dissociated from the spheroids. Therefore the results suggested that both spheroids and spheroid derived hMSCs may be more effective than hMSCs from adherent cultures in modulating inflammatory reactions. The suggestion was confirmed by the demonstration that the spheroids and spheroid derived hMSCs were more effective in suppressing TNFα production by LPS stimulated macrophages in culture. In addition. they were more effective in suppressing inflammation in an in vivo model for zymosan induced peritonitis. Also of special interest was that the spheroid hMSCs expressed high levels of transcripts for the tumor suppressor protein IL-24, an observation made previously with 3D cultures of hMSCs prepared using spinner flasks and a rotating wall vessel bioreactor (Frith, 2010). In addition, the spheroid hMSCs prepared under the conditions optimized to express TSG-6 also expressed high levels of transcripts for TRAIL that is selective for killing certain cancer cells (Mahmood, et al., Exp. Cell Res., Vol. 316, pgs. 887-899 (2010); Mellier, et al, Mol. Aspects Med., Vol. 31, pgs. 93-112 (2010)) and for CD82 that suppresses some metastases (Smith, et al., Nat. Rev. Cancer, Vol. 9, pgs 253-264 (2009)). The increased expression of TRAIL on the surface of the spheroid derived MSCs is important because TRAIL expressed on the surface of cells has shown to be far more effective in killing cancer cells than the soluble forms of the protein that have been in clinical trials. Therefore, hMSC spheroids and spheroid derived hMSCs may be particularly effective as an adjunct therapy for some types of cancers, particularly for therapy of cancers sensitive to anti-inflammatory agents such as aspirin or steroids (Grivennikov, et al., Cell, Vol, 140, pgs. 883-889 (2010)). A further advantage of the spheroid hMSCs was that they were less than one-fourth the volume of hMSCs from adherent cultures. Therefore a significantly smaller number was trapped in the lung after i.v. infusion and thus larger numbers were found in many tissues (Lee Cell Stem Cell, 2009; Lee, Blood, 2009).

The molecular forces that increase expression of anti-inflammatory and anti-tumorigenic genes in hMSCs assembled into spheroids are intriguing but unclear. Cells in spheroids are in close association with each other and probably signal cues to each other much easier than in monolayer cultures, where only a very small part of the cell can touch another cell and secreted molecules must be present in high amounts to ensure communication. The changes in the hMSCs as they form spheroids are probably the result of the non-adherent culture conditions, high degree of confluency, nutrient deprivation, air-liquid interface, and “microgravity” Of hanging drops. More detailed studies of each of these and other possible factors must be conducted to have a better understanding of the changes hMSCs accrue when they aggregate into spheroids.

The results presented here indicated that hMSCs can be activated non-chemically in hanging drops to secrete substantial quantities of potent anti-inflammatory proteins and express anti-tumorigenic molecules. Therefore spheroid hMSCs may have advantages for many therapeutic applications. In addition, hMSCs dissociated from spheroids provide extremely small activated cells that could have major advantages for i.v. administration.

### Example 4

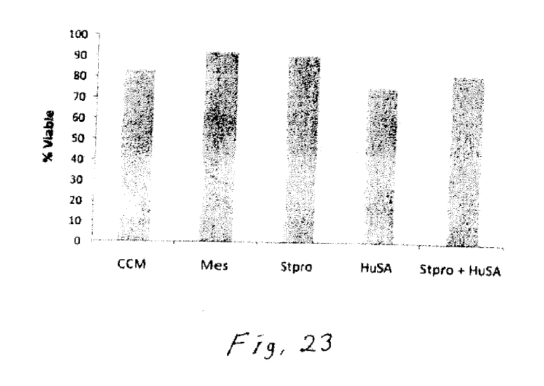

Spheroids of human mesenchymal stem cells were cultured in complete culture medium (CCM) containing α-MEM with 17% FRS, human serum albumin (HuSA) (Baxter Healthcare) Stem Pro Xeno-free Medium (Stpro) (Gibco), or Mesencult Xeno-free Medium (Stem Cell Technologies), or Mes. After 3 days, the spheroids were dissociated using trypsin/EDTA, and then were frozen in dimethylsulfoxide (DMSO). The cells then were thawed and the viability of the cells was determined by flow cytometry, measuring PI uptake and annexin V-FITC cell surface labeling. Unlabeled cells were considered viable. As shown in FIG. 23, the culturing of spheroids of mesenchymal stem cells in different media does not affect their viability.

### Example 5

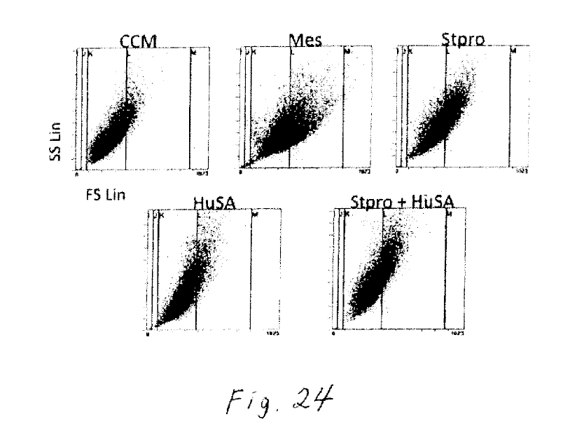

Spheroids of human mesenchymal stem cells were cultured for 3 days in CCM or in various commercially available Xeno-free media as described in Example 4. The spheroids then were dissociated using trypsin/EDTA, and then were frozen in DMSO. After a minimum of 1 week, the cells were thawed and then labeled with the viability dyes calcein AM and 7AAD to exclude dead cells from the analysis. Representative linear scatter plots determined through flow cytometric determination of the viable population are shown in FIG. 24. Size was quantified by comparing forward scatter (FS) properties of the cells with beads of known diameter (3, 7, 15, and 25 μm). Brackets were applied to the scatter plot at locations corresponding to the appropriate bead size. (I=0, J=3, K=7, L=15, M=25 μm). FIG. 24 shows that the mesenchymal stem cells dissociated from spheroids are larger, as shown by greater forward scattering of light (FS Lin) when the spheroids are cultured in media that do not contain fetal calf serum (FCS) that is found in the complete culture medium (CCM).

### Example 6

Spheroids of human mesenchymal stem cells were cultured for 3 days in CCM or in various commercially available Xeno-free media as described in Example 4, Expression of genes encoding the anti-inflammatory proteins TSG-6, STC-1, and growth differentiation factor-15 (GDF-15) then was analyzed by real-time RT PCR. Values are expressed as mean RQ±SD (n=3) as compared to an Adh Low sample.

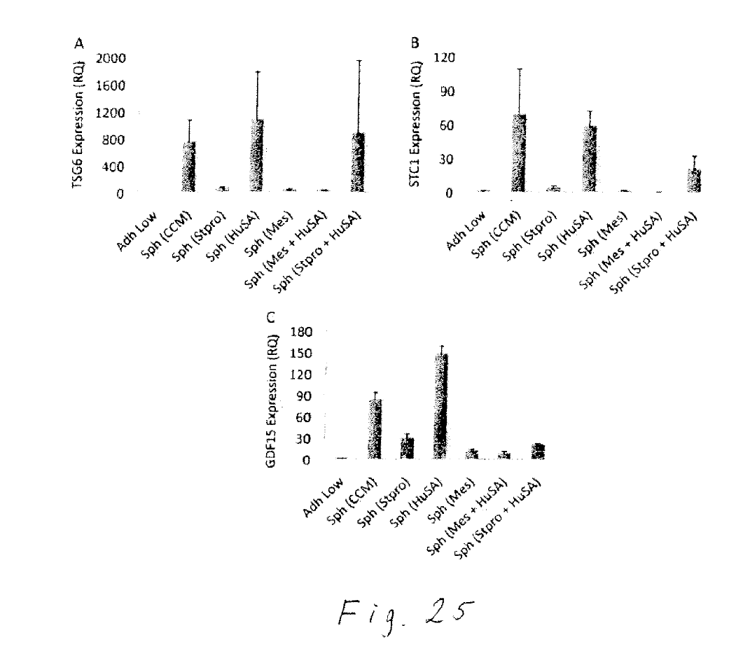

As shown in FIG. 25, the therapeutic proteins TSG-6, STC-1, and GM-15 are not expressed when the spheroids are cultured in media that does not contain fetal calf serum; however, the addition of human serum albumin (HuSA) increases expression of such proteins.

### Example 7

Spheroids of human mesenchymal stem cells were cultured for 3 days in the commercially available Xeno-free media described in Example 4. The media conditioned by the spheroids (conditioned medium, or CM) were collected, diluted 1:50, and added to mouse macrophages in the presence of 100 ng/ml LPS. Macrophages cultured with LPS (sMO) or without LPS (MO), and with non-conditioned media in the presence of LPS, served as controls. After 18 hours, the macrophage media were harvested and assayed for mTNFα by ELISA. Values are mean±SD (n=3).

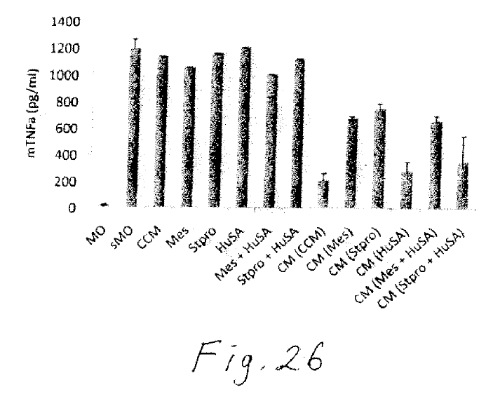

The results, as shown in the 6 columns on the right of FIG. 26, show that conditioned media (CM) from spheroids prepared in media containing fetal calf serum (i.e., CCM) are highly effective in inhibiting the production of TNFα. Conditioned media from spheroids cultured in the other media were effective in inhibiting TNFα, but to a lesser and more variable extent.

The disclosures of each and every patent, patent application, and publication cited herein are hereby incorporated herein by reference in their entireties.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. Thus, the invention may be practiced other than as particularly described and still be within the scope of the accompanying claims.

What is claimed is:

1. Mesenchymal stem cells in a spheroidal aggregate or mesenchymal stem cells obtained from a spheroidal aggregate, wherein said mesenchymal stem cells express increased amounts of at least one therapeutic protein compared to mesenchymal stem cells cultured as a monolayer.

2. The cells of claim 1 wherein said at least one therapeutic protein is selected from the group consisting of anti-inflammatory proteins, anti-apoptotie proteins, proteins that regulate cell growth and development, proteins that regulate an immune response, proteins that regulate hemotopoiesis, proteins which inhibit, prevent, or destroy the growth of tumors, proteins that regulate the homing of cells, proteins that are involved in cell adhesion and cell signaling, proteins that enhance angiogenesis, and combinations thereof.

3. The cells of claim 2 wherein said therapeutic protein is an anti-inflammatory protein.

4. The cells of claim 3 wherein said anti-inflammatory protein is TSG-6.

5. The cells of claim 2 wherein said protein is an anti-apoptotie protein.

6. The cells of claim 5 wherein said anti-apoptotic protein is STC-1.

7. The cells of claim 2 wherein said protein is a protein that regulates cell growth and development.

8. The cells of claim 7 wherein said protein is LIF.

9. The cells of claim 2 wherein said protein is a protein that regulates hematopoiesis.

10. The cells of claim 9 wherein said protein is IL-11.

11. The cells of claim 2 wherein said protein inhibits, prevents, or destroys the growth or tumors.

12. The cells of claim 11 wherein said protein is TNF-α related apoptosis inducing ligand.

13. The cells of claim 11 wherein said protein is IL-24.

14. The cells of claim 11 wherein said protein is CD82.

15. The cells of claim 2 wherein said protein regulates homing of cells.

16. The cells of claim 15 wherein said protein is CXCR4.

17. The cells of claim 2 wherein said protein is a protein involved in cell adhesion and cell signaling.

18. The cells of claim 17 wherein said protein is ITGA2.

19. The cells of claim 2 wherein said protein enhances angiogenesis.

20. The cells of claim 19 wherein said protein is IL-8.

21. A method of treating inflammation in a patient, comprising:
administering to said patient mesenchymal stem cells in a spheroidal aggregate or mesenchymal stem cells obtained from a spheroidal aggregate, wherein said mesenchymal stem cells express increased amounts of an anti-inflammatory protein compared to mesenchymal stem cells cultured as a monolayer, wherein said mesenchymal stem cells are administered in an amount effective to treat said inflammation in said patient.

22. The method of claim 21 wherein said anti-inflammatory protein is TSG-6.

23. A method of treating a tumor in a patient comprising:
administering to said patient mesenchymal stem cells in a spheroidal aggregate or mesenchymal stem cells obtained from a spheroidal aggregate wherein said mesenchymal stem cells express increased amounts of a protein that inhibits, prevents, or destroys the growth of tumors compared to mesenchymal stem cells cultured as a monolayer, wherein said mesenchymal stem cells are administered in an amount effective to inhibit, prevent, or destroy the growth of a tumor in said patient.

24. The method of claim 23 wherein said protein which inhibits, prevents, or destroys the growth of a tumor is TNF-α related apoptosis inducing ligand.

25. The method of claim 23 wherein said protein which inhibits, prevents, or destroys the growth of a tumor is IL-24.

26. A method of regulating an immune response in a patient, comprising:
administering to said patient mesenchymal stem cells in a spheroidal aggregate or mesenchymal stem cells obtained from a spheroidal aggregate, wherein said mesenchymal stem cells express increased amounts of a protein that regulates an immune response compared to mesenchymal stem cells cultured as a monolayer, wherein said mesenchymal stem cells are administered in an amount effective to regulate an immune response in said patient.

27. A method of producing a spheroidal aggregate of mesenchymal stem cells, comprising:
culturing said mesenchymal stem cells in a medium including a serum selected from the group consisting of fetal bovine serum and horse serum.

28. The method of claim 27 wherein said serum is fetal bovine serum.

29. The method of claim 28 wherein said fetal bovine serum is present in said medium in an amount of up to about 20%.

30. The method of claim 29 wherein said fetal bovine serum is present in said medium in an amount of about 17%.

31. The method of claim 27 which said mesenchymal stem cells are cultured in said medium as hanging drops of mesenchymal stem cells.

32. The method of claim 31 wherein each hanging drop of mesenchymal stem cells contains from about 10,000 to about 500,000 cells per drop.

33. The method of claim 32 wherein each hanging drop of mesenchymal stem cells contains from about 10,000 to about 250,000 cells per drop.

34. The method of claim 33 wherein each hanging drop of mesenchymal stem cells contains from about 10,000 to about 25,000 cells per drop.

35. The method of claim 34 wherein each hanging drop of mesenchymal stem cells contains about 25,000 cells per drop.

36. A method of providing a therapeutic effect in an animal comprising:
administering to said animal a composition comprising a medium in which there have been cultured previously spheroidal aggregates of mesenchymal stem cells, wherein said medium is present in said composition in an amount effective to provide a therapeutic effect in said animal.
